(19) United States
(12) Patent Application Publication
(10) Pub. No.: US 2015/0132342 A1
(43) Pub. Date: May 14, 2015

(54) NOVEL IMMUNE ACTIVATORS: SUBSTITUTE 4-AMINOQUINAZOLINES

(71) Applicant: The Regents of the University of California, Oakland, CA (US)

(72) Inventors: Dennis A. Carson, La Jolla, CA (US); Howard B. Cottam, La Jolla, CA (US); Tomoko Hayashi, La Jolla, CA (US); Afshin Nour, La Jolla, CA (US)

(21) Appl. No.: 14/536,146

(22) Filed: Nov. 7, 2014

Related U.S. Application Data
(60) Provisional application No. 61/902,016, filed on Nov. 8, 2013.

(51) Int. Cl. C07D 239/94; A61K 39/39

(52) U.S. Cl. CPC C07D 239/94; A61K 39/39
USPC 424/278.1; 544/293; 435/375

(57) ABSTRACT

Provided herein are compounds and methods for modulating an immune response in a subject in need thereof. Further provided herein are compounds and methods for modulating a toll-like receptor protein.

### CROSS-REFERENCES TO RELATED APPLICATIONS

This application claims the benefit of U.S. Provisional Application No. 61/902,016, filed Nov. 8, 2013, which is herein incorporated in its entirely and for all purposes.

### STATEMENT AS TO RIGHTS TO INVENTIONS MADE UNDER FEDERALLY SPONSORED RESEARCH AND DEVELOPMENT

This invention was made with government support under Grant No. HHSN272200900034C awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 88654-918866_ST25.TXT, created on Nov. 7, 2014, 75,275 bytes, machine format IBM-PC, MS-Windows operating system, is hereby incorporated by reference.

### BACKGROUND OF THE INVENTION

The innate immune response is the first line of defense against microbial pathogens such as viruses, bacteria, fungi, and protozoa. One component of the innate immune response is the NF_{κ}B family of transcription factors. The Toll-like receptors (TLRs) are known components of the innate immune system that regulate NF_{κ}B activation. In general, the TLRs recognize macromolecules that are associated with pathogens and with cell stress. These pathogen-associated molecular patterns (PAMPs) and their corresponding TLRs include: lipopeptides (TLR2), double-stranded RNA (TLR3), lipopolysaccharide (LPS) (TLR4), bacterial flagellin (TLR5), guanine and uridine-rich single-stranded RNA (TLR7, 8), and hypo-methylated CpG rich DNA (TLR9). Adjuvants are added to antigens in vaccines to augment adaptive immune responses to poorly immunogenic antigens, and to increase protective antibody titers in at risk populations due to age (infants and the elderly), or disease (diabetes, liver failure). Adjuvants also facilitate the use of smaller doses of antigen, and enable effective immunization with fewer booster immunizations. Thus, there is a need in the art for small molecular weight ligands as adjuvants or immunotherapeutic agents which preferentially stimulate human innate immune cells. Provided herein are solutions to these and other problems in the art.

### BRIEF SUMMARY OF THE INVENTION

Provided herein are compounds having the formula:

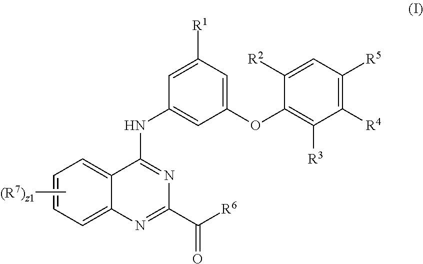

or a pharmaceutically acceptable salt thereof.

In one aspect, R¹ and R² are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³, R⁴ and R⁵ are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or wherein R³ and R⁴ are optionally joined together to form a substituted or unsubstituted cycloalkyl, substituted or unsubstituted heterocycloalkyl, substituted or unsubstituted aryl or substituted or unsubstituted heteroaryl, or wherein R⁴ and R⁵ are optionally joined together to form a substituted or unsubstituted cycloalkyl, substituted or unsubstituted heterocycloalkyl, substituted or unsubstituted aryl or substituted or unsubstituted heteroaryl. R⁶ is —OR⁹, —N(R⁸)(R⁹), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is independently halogen, —CN, —SH, —OH, —COOH, —NH₂, —CONH₂, —NO₂, —CF₃, —CCl₃, -L¹-R¹³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is independently hydrogen or substituted or unsubstituted alkyl. R⁹ is independently hydrogen, substituted or unsubstituted alkyl, or a hydrophilic polysaccharide. L¹ is substituted or unsubstituted alkylene or substituted or unsubstituted heteroalkylene. R¹³ is halogen, —CN, —SH, —OH, —COOH, —NH₂, —CONH₂, —NO₂, —CF₃, —CCl₃, substituted or unsubstituted cycloalkyl, substituted or unsubstituted heterocycloalkyl, substituted or unsubstituted aryl, or substituted or unsubstituted heteroaryl. The symbol z1 is an integer from 0 to 4.

Also provided herein are pharmaceutical compositions. In one aspect is a pharmaceutical composition that includes a compound described herein and a pharmaceutically acceptable excipient. In another aspect is a pharmaceutical composition that includes an antigen and a compound described herein (e.g. a vaccine pharmaceutical composition). Further provided herein are vaccine compositions. In one aspect is a vaccine composition that includes a compound described herein and an antigen. In another aspect is a vaccine composition that includes a compound described herein, an antigen, and an adjuvant.

Provided herein are biological cells, where the biological cells include a compound described herein. Provided herein are mixtures that include a compound described herein. In one aspect is a mixture that includes a compound described herein and a TLR modulator.

Methods of modulating a Toll-like receptor protein are provided herein. In one aspect is a method of modulating a TLR protein by contacting the TLR protein with a compound described herein. In another aspect is a method of modulating a TLR4 protein by contacting the TLR4 protein with a compound described herein.

Also provided herein are methods of treating or preventing a disease in a subject in need thereof. In one aspect is a method of treating or preventing a disease in a subject in need thereof by administering an effective amount of a compound described herein to modulate an immune response (e.g. by modulating a TLR protein on a cell) in the subject. In another aspect is a method of treating a disease in a subject in need thereof by administering a therapeutically effective amount of a compound described herein to modulate an immune response in the subject. In yet another aspect is a method of preventing a disease in a subject in need thereof by administering a prophylactically effective amount of a compound described herein to modulate an immune response in the subject.

### BRIEF DESCRIPTION OF THE DRAWINGS

FIG. 1. A) Compound 1a is a TLR4 specific ligand that activates NFκB in a TLR4/MD-2-dependent, but CD14-independent manner. B) Human TLR2, TLR3, TLR4/MD-2/CD14, TLR5, TLR7, TLR8, and TLR9 HEK 293 Blue cells or NFκB/SEAPorter cells were incubated with compound 1a (10 μM) for 20-24 h, and activation was evaluated by SEAP secretion in the culture supernatants using SEAPorter assay kit. Data shown are mean±SEM of triplicates and representative of two to three independent experiments showing similar results. * denotes p<0.05 was considered significant compared to the vehicle control using Student's t test. (B-D) mBMDC prepared from wild type mice or mice genetically deficient for B) TLR4, C) MD-2 or CD14 were stimulated with Compound 1a (10 μM). IL-6 levels in the culture supernatant were determined by ELISA. D) Human TLR4 transfectoma cells were incubated with 10 μM Compound 1a or 10 ng/mL LPS in the presence or absence of TLR4 antagonist LPS-RS (12, 111, 1000 ng/mL). Activation of the TLR4/NF-κB pathway was evaluated by SEAP secretion in the culture supernatants. E) Human PBMC were incubated with Compound 1a alone or Compound 1a with polymyxin B (10 μg/mL) overnight. IL-8 in the culture supernatants was measured by ELISA. Data shown are mean±SEM of triplicates and representative of two independent experiments showing similar results. F) The same concentration of polymyxin B as in E) significantly suppressed IL-8 release by LPS stimulation. * denotes p<0.05 considered as significant compared to vehicle using one way ANOVA with Dunnett's post hoc testing.

FIG. 2. Compound 1a induces type I IFN and expression of co-stimulatory molecules. A) Human PBMC were incubated with compound 1a (10 μM), LPS (10 ng/mL), or vehicle overnight. The release of type I IFN in the culture supernatants was measured by luciferase release in L929-ISRE reporter cells. Data shown are mean±SEM of triplicates. B) Compound 1a- or vehicle-treated wild type or Tlr4^{-/-}mBMDC were stained for CD11c and either CD40, CD80, or CD86. The expression levels of CD40, CD80, or CD86 were evaluated in the gated CD11c⁺ population. DMSO 0.5% served as the vehicle control. C) Human PBMC were incubated with compound 1a (5 μM) overnight. CD80 and CD83 expression in gated CD11c⁺ HLA-DR⁺ population was analyzed by flow cytometer. Data are representative of two independent experiments showing similar results.

FIG. 3. Compound 1a is more potent in human cells. A) CellSensor® RAW264.7 or CellSensor® THP-1 cells were incubated with graded concentrations of Compound 1a. The levels of NFκB activation were normalized with LPS and expressed as percent activation. The average response ratio of LPS (5 ng/mL) in RAW and THP-1 CellSensor® cells was 6.5±0.17 and 3.27±0.67, respectively. B) Murine BMDC or C) hPBMC were stimulated with graded concentrations of Compound 1a overnight. Murine IL-6 or hIL-8 in the culture supernatant was determined by ELISA. D) Histogram of IL-6 levels (ng/mL) demonstrating increased production in WT and hTLR4 Tg after LPS administration relative to compound 1a. E) Histogram of IP-10 levels (ng/mL) demonstrating increased production in WT and hTLR4 Tg after LPS administration relative to compound 1a. Data shown are mean±SEM of triplicates and representative of two independent experiments showing similar results.

FIG. 4. Predicted binding mode of compound 1a to human TLR4/MD-2 complex. The compound has attractive interactions with the seven residues of MD-2, and three residues of TLR-4 which significantly increase the probability and stability of binding. SEQ ID NOS:10-11 employ the sequence numbering protocol of Protein Data Bank entry 3fxi; compare with SEQ ID NO:3 for TLR4.

FIG. 5. Predicted binding interactions of compound 1a with hTLR4/MD-2 complex (PDB ID: 3fxi) (SEQ ID NOS:10-11). SEQ ID NOS:10-11 employ the sequence numbering protocol of Protein Data Bank entry 3fxi; compare with SEQ ID NO:3 for TLR4. The set of H-bond and hydrophobic interactions supports the prediction of the compound position in the pocket.

FIG. 6. Representative histograms of results of substituted 4-aminoquinazolines using hTLR4 transfectoma and primary hPBMC. A) and B) show human TLR4 transfectoma cells were incubated with graded concentrations of 1a or indicated SAR compounds (compounds 1e and 1g; compounds 11a and 13a) for 20-22 h. Release of SEAP was measured by OD₆₃₀ in a QUANTI-BLUE™ assay. C) and D): Human PBMC from two independent donors were stimulated with 0.5 or 5 μM1a overnight. IL-8 in the culture supernatant was determined by ELISA. Data shown are mean±SEM of triplicate and represents two to three experiments showing similar results.

### DETAILED DESCRIPTION OF THE INVENTION

The abbreviations used herein have their conventional meaning within the chemical and biological arts. The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, B, As, and Si may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH—CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heteroalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, aryl is a phenyl or naphthyl. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, or quinolyl (including embodiments and all attachment positions e.g. 1-naphthyl or 2-naphthyl) Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R”, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R”, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. The ring-forming substituents may be attached to adjacent members of the base structure. For example, two ring-forming substituents attached to adjacent members of a cyclic base structure create a fused ring structure. The ring-forming substituents may be attached to a single member of the base structure. For example, two ring-forming substituents attached to a single member of a cyclic base structure create a spirocyclic structure. The ring-forming substituents may be attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

Each substituted group described in the compounds herein may be substituted with at least one substituent group. More specifically, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein may be substituted with at least one substituent group. At least one or all of these groups may be substituted with at least one size-limited substituent group. At least one or all of these groups may be substituted with at least one lower substituent group.

The compounds herein may each be substituted or unsubstituted alkyl, a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. Each substituted or unsubstituted alkylene may be a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

Each substituted or unsubstituted alkyl may be a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. Each substituted or unsubstituted alkylene may be a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds, generally recognized as stable by those skilled in the art, are within the scope of the invention. Further, unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents arepresent, each R¹³ substituent may be distinguished as R^{13.1}, R^{13.2}, R^{13.3}, R^{13.4}, etc., wherein each of R^{13.1}, R^{13.2}, R^{13.3C}, R^{13.4}, etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts. Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art. As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein include those compounds that readily undergo chemical or enzymatic changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. A patient may be a human. A patient may be a domesticated animal. A patient may be a dog. A patient may be a parrot. A patient may be a livestock animal. A patient may be a mammal. A patient may be a cat. A patient may be a horse. A patient may be a bovine A patient may be a canine A patient may be a feline. A patient may be an ape. A patient may be a monkey. A patient may be a mouse. A patient may be an experimental animal. A patient may be a rat. A patient may be a hamster. A patient may be a test animal. A patient may be a newborn animal. A patient may be a newborn human. A patient may be a newborn mammal. A patient may be an elderly animal. A patient may be an elderly human. A patient may be an elderly mammal. A patient may be a geriatric patient.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof. A “reduction” of a symptom or symptoms (and grammatical equivalents of this phrase) means decreasing of the severity or frequency of the symptom(s), or elimination of the symptom(s). A “prophylactically effective amount” of a composition (vaccine) is an amount of a composition that, when administered to a subject, will have the intended prophylactic effect, e.g., preventing or delaying the onset (or reoccurrence) of an injury, disease), pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses (e.g. prime-boost). Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. Preparation also refers to formulation of the compounds described herein for administration as a vaccine.

As used herein, the term “administering” refers to oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by a route acceptable for the chosen formulation and can include parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The compositions described herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions described herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). The formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

Pharmaceutical compositions may include compositions wherein the active ingredient (e.g. compounds described herein) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. When administered in methods to prevent (e.g. prophylactically treat) a disease, such compositions will contain an amount of active ingredient effective to prevent the activity of a target molecule or prevent or reduce the occurrence of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The compounds described herein can be used in combination with one another, with other active drugs known to be useful in treating a disease (e.g. anti-cancer drugs or anti-inflammatory drug) or with adjunctive agents that may not be effective alone, but may contribute to the efficacy of the active agent. Thus, the compounds described herein may be co-administered with one another or with other active drugs known to be useful in treating a disease.

The compounds described herein may be used in combination with one or more adjuvants known to be useful in eliciting immune response or enhancing an antigenic response of an administered vaccine.

Vaccine compositions can be delivered intramuscularly or subcutaneously. An “administration device” as used herein refers to a device used to administer the vaccine compositions described herein. Administration devices include, but are not limited, to devices for oral, topical, inhalation, or injection of the vaccine compositions described herein. Exemplary administration devices include, but are not limited to, ampoules, droppers, patches, sprays, pumps, IV lines, syringes.

By “co-administer” it is meant that a compound described herein is administered at the same time, just prior to, or just after the administration of one or more additional therapies, for example, an anti-cancer agent as described herein. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances (e.g. anticancer agents). Co-administration also includes administration at the same time, just prior to, or just after the administration of one or more adjuvants in a vaccine.

Co-administration includes administering one active agent (e.g. a complex described herein) within 0.5, 1, 2, 4, 6, 8, 10, 12, 16, 20, or 24 hours of a second active agent (e.g. anti-cancer agents). Co-administration may include administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. The active agents can be formulated separately. The active and/or adjunctive agents may be linked or conjugated to one another. The compounds described herein may be combined with treatments for cancer such as chemotherapy or radiation therapy. Co-administration also includes administration of vaccine compositions using a prime-boost method of administration.

The term “prime-boost” or “prime boost” as applied to a methodology of administering vaccines is used according to its plain ordinary meaning in virology and immunology and refers to a method of vaccine administration in which a first dose of a vaccine or vaccine component is administered to a subject or patient to begin the administration (prime) and at a later time (e.g. hours, days, weeks, months later) a second vaccine is administered to the same patient or subject (boost). The first and second vaccines may be the same or different but are intended to both elicit an immune response useful in treating or preventing the same disease or condition (e.g. cancer, an autoimmune disease, an infectious disease, or inflammation).

The term “associated” or “associated with” in the context of a substance or substance activity or function associated with a disease means that the disease is caused by (in whole or in part), a symptom of the disease is caused by (in whole or in part) the substance or substance activity or function, or a side-effect of the compound (e.g. toxicity) is caused by (in whole or in part) the substance or substance activity or function.

“Disease” or “condition” refer to a state of being or health status of a patient or subject capable of being treated with the compounds or methods provided herein. Disease as used herein may refer to cancer, an infectious disease, an autoimmune disease, or an inflammatory disease or inflammation associated with a disease (e.g. cancer or infectious disease).

As used herein, an “autoimmune disease” refers to a disease or disorder that arises from altered immune reactions by the immune system of a subject, e.g., against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, vitiligo, asthma, and allergic asthma.

As used herein, the term “cancer” refers to all types of cancer, neoplasm, or malignant or benign tumors found in mammals, including leukemia, carcinomas and sarcomas. Exemplary cancers include acute myeloid leukemia (“AML”), chronic myelogenous leukemia (“CML”), and cancer of the brain, breast, pancreas, colon, liver, kidney, lung, non-small cell lung, melanoma, ovary, sarcoma, and prostate. Additional examples include, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The murine leukemia model is widely accepted as being predictive of in vivo anti-leukemic activity. It is believed that a compound that tests positive in the P388 cell assay will generally exhibit some level of anti-leukemic activity regardless of the type of leukemia being treated. Accordingly, the present invention includes a method of treating leukemia, including treating acute myeloid leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

“Anti-cancer agent” is used in accordance with its plain and ordinary meaning and refers to a composition (e.g. compound, drug, antagonist, inhibitor, modulator) having antineoplastic properties or the ability to inhibit the growth or proliferation of cells. An anti-cancer agent may be chemotherapeutic. An anti-cancer agent may be an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin Al (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

“Cancer model organism”, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The term “antibody” or “antibodies” as used herein refers to all types of immunoglobulins, including IgG, IgM, IgA, IgD, and IgE, and any sub-isotype, including IgG1, IgG2a, IgG2b, IgG2c, IgG3 and IgG4, IgE1, IgE2, etc., and may include Fab or antigen-recognition fragments thereof. The antibodies may be monoclonal or polyclonal and may be of any species of origin, including e.g., mouse, rat, rabbit, horse, or human, or may be chimeric antibodies. See, e.g., M. Walker et al., Molec. Immunol. 1989, 26:403-11; Morrision et al., Proc. Nat'l. Acad. Sci., 1984, 81:6851; Neuberger et al, Nature, 1984, 312:604. The antibodies may be recombinant monoclonal antibodies produced according to the methods disclosed in U.S. Pat. No. 4,474,893 or U.S. Pat. No. 4,816,567. The antibodies may also be chemically constructed by specific antibodies made according to the method disclosed in U.S. Pat. No. 4,676,980.

The term “vaccine” is used according to its plain ordinary meaning within medicine and immunology and refers to a composition including an antigenic component for administration to a subject (e.g. human), which elicits an immune response to an antigenic component. A vaccine may be therapeutic. A vaccine may be prophylactic. A vaccine may include one or more adjuvants.

The term “adjuvant” (also referred to herein as a “vaccine adjuvant”) is used in accordance with its plain ordinary meaning within immunology and refers to a substance that is commonly used as a component of a vaccine. Adjuvants may increase an antigen specific immune response in a subject when administered to the subject with one or more specific antigens as part of a vaccine. An adjuvant may accelerate an immune response to an antigen. An adjuvant may prolong an immune response to an antigen. An adjuvant may enhance an immune response to an antigen. Exemplary adjuvants include, but are not limited to, an aluminum-based mineral salt adjuvant (e.g. Alum aluminum hydroxide gel, Imject Alum Adjuvant™, Adju-Phos™, Alhydrogel™, amorphous or crystalline aluminum hydroxide, aluminum oxyhydroxide, aluminum hydroxycarbonate, or amorphous aluminum hydroxyphosphate), squalene, bacterial cell wall components, molecular cages, a nucleic acid, an oil, a virosome, QS21, or MF59.

The term “antigen” refers, in the usual and customary sense, to a substance that binds specifically to an antibody or that can be recognized by antigen receptors (e.g., B-cell receptor, T-cell receptor and the like) of the adaptive immune system thereby eliciting an immunological response. The terms “antigenic” or “antigenicity” are used in accordance with their plain ordinary meaning within immunology and refer an immunological response elicited from an antigen. Thus, “antigenic” and “antigenicity” may refer to specific immunological response from a specified subject. The antigenicity may be associated with an immunological response from a bird, reptile, or fish. Antigenicity may be associated with immunological response from a mammal such as, for example, a mouse, rat, guinea pig, rabbit, cow, dog, cat, chimpanzee or human. Antigenicity may be associated with immunological response from a mouse, rat, guinea pig, or rabbit. Antigenicity may be associated with immunological response from a human. The term “antigenic site” therefore refers to a residue of—, a modification of—, a portion of—, or the full-length of—a polynucleotide or polypeptide that elicits an immunological response as described above. An antigenic site may be a polypeptide sequence or amino acid residues within a TLR described herein.

The terms “immune response” and the like refer, in the usual and customary sense, to a response by an organism that protects against disease. The response can be mounted by the innate immune system or by the adaptive immune system, as well known in the art. The terms “modulating immune response” and the like refer to a change in the immune response of a subject as a consequence of administration of an agent, e.g., a compound described herein. The term “modulating” as used herein refers to either increasing or decreasing the level of activity of the modulated entity, e.g., immune response. Accordingly, an immune response can be activated or deactivated as a consequence of administration of an agent, e.g., a compound described herein. The term “activated” means an enhancement in the activity of the activated entity. The term “deactivated” means a diminution in the activity of the deactivated entity. A deactivated immune response may be measurable, albeit at a reduced level compared to levels absent deactivation.

The term “TLR” refers generally to Toll-like receptors which are are critical components of the innate immune system that regulate NF_{κ}B activation, as well known in the art. A “TLR modulator,” “TLR immunomodulator” and the like as used herein refer, in the usual and customary sense, to compounds which agonize or antagonize a Toll Like Receptor. See e.g., PCT/US2010/000369, Hennessy, E. J., et al., Nature Reviews 2010, 9:283-307; PCT/US2008/001631; PCT/US2006/032371; PCT/US2011/000757. Accordingly, a “TLR agonist” is a TLR modulator which agonizes a TLR, and a “TLR antagonist” is a TLR modulator which antagonizes a TLR. The term “TLR2” as used herein refers to the product (NCBI Accession AAH33756.1, SEQ ID NO:1) of the TLR2 gene, and homologs and functional fragments thereof. The term “TLR3” as used herein refers to the product (NCBI Accession ABC86910.1, SEQ ID NO:2) of the TLR3 gene, and homologs and functional fragments thereof. The term “TLR4” as used herein refers to the product of the TLR4 gene, and homologs, isoforms, and functional fragments thereof: Isoform 1 (NCBI Accession NP_{—}612564.1, SEQ ID NO:3); Isoform 2 (NCBI Accession NP_{—}003257.1, SEQ ID NO:4); Isoform 3 (NCBI Accession NP_{—}612567.1, SEQ ID NO:5). The term “TLR5” as used herein refers to the product (NCBI Accession AAI09119, SEQ ID NO:6) of the TLR5 gene, and homologs, and functional fragments thereof. The term “TLR7” as used herein refers to the product (NCBI Accession AAZ99026, SEQ ID NO:7) of the TLR7 gene, and homologs, and functional fragments thereof. The term “TLR8” as used herein refers to the product (NCBI Accession AAZ95441, SEQ ID NO:8) of the TLR8 gene, and homologs, and functional fragments thereof. The term “TLR9” as used herein refers to the product (NCBI Accession AAZ95520, SEQ ID NO:9) of the TLR9 gene, and homologs, and functional fragments thereof.

The terms “inflammation” and the like refer, in the usual and customary sense, to the pain, heat, erythema, swelling and/or loss of function that accompanies the complex biological response of tissues (e.g., vascular tissue) to harmful stimuli (e.g., pathogen invasion, damage to cells, irritants, and the like). Mediators of inflammation include plasma derived mediates such as bradykinin, C3, C5a, Factor XII, membrane attack complex, plasmin, and thrombin, as known in the art. Cell derived mediators of inflammation include lysosome granulates, histamine, IFN-γ, IL-8, leukotriene B4, nitric oxide, prostaglandins, TNF-α and IL-1. Accordingly, the terms “decrease inflammation” and the like mean that inflammation, as judged by assays well known in the art, is reduced relative to that observed in the absence of the compound described herein.

### I. COMPOSITIONS

Provided herein are compounds having the formula:

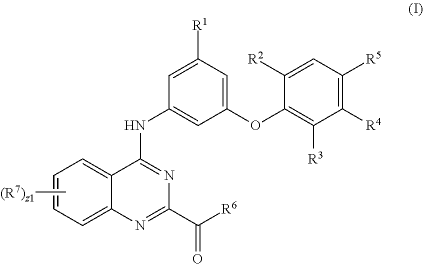

In a first aspect, R¹, R², R³, R⁴ and R⁵ are each independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is —OH, —N(R⁸)(R⁹), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is independently halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is independently hydrogen or substituted or unsubstituted alkyl. R⁹ is independently hydrogen, substituted or unsubstituted alkyl, or a hydrophilic polysaccharide. The symbol z1 is an integer from 0 to 4.

In a second aspect, R¹ and R² are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³, R⁴ and R⁵ are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or wherein R³ and R⁴ are optionally joined together to form a substituted or unsubstituted cycloalkyl, substituted or unsubstituted heterocycloalkyl, substituted or unsubstituted aryl or substituted or unsubstituted heteroaryl, or wherein R⁴ and R⁵ are optionally joined together to form a substituted or unsubstituted cycloalkyl, substituted or unsubstituted heterocycloalkyl, substituted or unsubstituted aryl or substituted or unsubstituted heteroaryl. R⁶ is —OR⁹, —N(R⁸)(R⁹), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is independently halogen, —CN, —SH, —OH, —COOH, —NH₂, —CONH₂, —NO₂, —CF₃, —CCl₃, -L¹-R¹³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is independently hydrogen or substituted or unsubstituted alkyl. R⁹ is independently hydrogen, substituted or unsubstituted alkyl, or a hydrophilic polysaccharide. L¹ is substituted or unsubstituted alkylene or substituted or unsubstituted heteroalkylene. R¹³ is substituted or unsubstituted cycloalkyl, substituted or unsubstituted heterocycloalkyl, substituted or unsubstituted aryl, or substituted or unsubstituted heteroaryl. The symbol z1 is an integer from 0 to 4.

R¹ may be hydrogen, halogen, —CN, —SH, —OH, —COOH, —NH₂, —CONH₂, —NO₂, —CF₃, —CCl₃, substituted or unsubstituted alkyl, or substituted or unsubstituted heteroalkyl. R¹ may be substituted or unsubstituted cycloalkyl, substituted or unsubstituted heterocycloalkyl, substituted or unsubstituted aryl, or substituted or unsubstituted heteroaryl. R¹ may be hydrogen. R¹ may be halogen. R¹ may be —CN. R¹ may be —SH. R¹ may be —OH. R¹ may be —COOH. R¹ may be —NH₂. R¹ may be —CONH₂. R¹ may be —NO₂. R¹ may be —CF₃ or —CCl₃. R¹ may be substituted or unsubstituted alkyl. R¹ may be substituted or unsubstituted heteroalkyl. R¹ may be substituted alkyl. R¹ may be unsubstituted alkyl. R¹ may be substituted or unsubstituted C₁-C₅ alkyl. R¹ may be substituted C₁-C₅ alkyl. R¹ may be substituted or unsubstituted C₁-C₃ alkyl. R¹ may be substituted C₁-C₃ alkyl. R¹ may be substituted heteroalkyl. R¹ may be unsubstituted heteroalkyl. R¹ may be substituted or unsubstituted 3 to 6 membered heteroalkyl. R¹ may be substituted 3 to 6 membered heteroalkyl.

R¹ may be hydrogen, halogen, —CN, —SH, —OH, —COOH, —NH₂, —CONH₂, —NO₂, —CF₃, —CCl₃, R^{1A}-substituted or unsubstituted alkyl, or R^{1A}-substituted or unsubstituted heteroalkyl. R¹ may be hydrogen, halogen, —CN, —SR^{1A}, —OR^{1A}, —COOR^{1A}, —NR^{1A}R^{1B}, —CONR^{1A}R^{1B}, —NO₂, —CF₃, —CCl₃, R^{1A}-substituted or unsubstituted alkyl, or R^{1A}-substituted or unsubstituted heteroalkyl. R¹ may be R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. R¹ may be R^{1A}-substituted or unsubstituted alkyl, or R^{1A}-substituted or unsubstituted heteroalkyl, R^{1A}-substituted or unsubstituted cycloalkyl, R^{1A}-substituted or unsubstituted heterocycloalkyl, R^{1A}-substituted or unsubstituted aryl, or R^{1A}-substituted or unsubstituted heteroaryl. R¹ may be hydrogen. R¹ may be R^{1A}-substituted or unsubstituted alkyl. R¹ may be R^{1A}-substituted or unsubstituted heteroalkyl. R¹ may be R^{1A}-substituted alkyl. R¹ may be R^{1A}-substituted or unsubstituted C₁-C₅ alkyl. R¹ may be R^{1A}-substituted C₁-C₅ alkyl. R¹ may be R^{1A}-substituted or unsubstituted C₁-C₃ alkyl. R¹ may be R^{1A}-substituted C₁-C₃ alkyl. R¹ may be R^{1A}-substituted heteroalkyl. R¹ may be R^{1A}-substituted or unsubstituted 3 to 6 membered heteroalkyl. R¹ may be R^{1A}-substituted 3 to 6 membered heteroalkyl. R¹ may be OR^{1A} or NR^{1A}R^{1B}, where R^{1A} and R^{1B} are independently hydrogen or substituted or unsubstituted alkyl. R¹ may be OR^{1A} or NR^{1A}R^{1B}, where R^{1A} and R^{1B} are independently hydrogen or methyl.

R^{1A} and R^{1B} are independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. R^{1A} and R^{1B} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl (e.g. C₁-C₃ alkyl), unsubstituted heteroalkyl (e.g. 2 to 5 membered heteroalkyl), unsubstituted (e.g. C₃-C₆ cycloalkyl), unsubstituted heterocycloalkyl (e.g. 3 to 6 membered heterocycloalkyl), unsubstituted aryl (e.g. phenyl), or unsubstituted heteroaryl (e.g. 5 or 6 membered heteroaryl). R^{1A} and R^{1B} may independently be halogen, —CF₃, —OH, —NH₂, unsubstituted alkyl (e.g. C₁-C₃ alkyl), unsubstituted heteroalkyl (e.g. 2 to 5 membered heteroalkyl), unsubstituted (e.g. C₃-C₆ cycloalkyl), unsubstituted heterocycloalkyl (e.g. 3 to 6 membered heterocycloalkyl), unsubstituted aryl (e.g. phenyl), or unsubstituted heteroaryl (e.g. 5 or 6 membered heteroaryl). R^{1A} and R^{1B} may independently be unsubstituted alkyl (e.g. C₁-C₃ alkyl), unsubstituted heteroalkyl (e.g. 2 to 5 membered heteroalkyl), unsubstituted cycloalkyl (e.g. C₃-C₆ cycloalkyl), unsubstituted heterocycloalkyl (e.g. 3 to 6 membered heterocycloalkyl), unsubstituted aryl (e.g. phenyl), or unsubstituted heteroaryl (e.g. 5 or 6 membered heteroaryl).

R² may be hydrogen, halogen, —CN, —SH, —OH, —COOH, —NH₂, —CONH₂, —NO₂, —CF₃, —CCl₃, substituted or unsubstituted alkyl or substituted or unsubstituted heteroalkyl. R² may be hydrogen. R² may be halogen. R² may be —CN, —SH, —OH, —COOH, —NH₂, —CONH₂, —CF₃, or —CCl₃. R² may be —NO₂. R² may be substituted or unsubstituted alkyl. R² may be substituted or unsubstituted heteroalkyl. R² may be hydrogen, substituted or unsubstituted alkyl, or substituted or unsubstituted heteroalkyl.

R² may be hydrogen or substituted or unsubstituted alkyl. R² may be hydrogen or substituted alkyl. R² may be hydrogen or unsubstituted alkyl. R² may be hydrogen or substituted or unsubstituted C₁-C₅ alkyl. R² may be hydrogen or substituted C₁-C₅ alkyl. R² may be hydrogen or unsubstituted C₁-C₅ alkyl. R² may be hydrogen or substituted or unsubstituted C₁-C₃ alkyl. R² may be hydrogen or substituted C₁-C₃ alkyl. R² may be hydrogen or unsubstituted C₁-C₃ alkyl. R² may be substituted or unsubstituted alkyl. R² may be substituted alkyl. R² may be unsubstituted alkyl. R² may be substituted or unsubstituted C₁-C₅ alkyl. R² may be substituted C₁-C₅ alkyl. R² may be unsubstituted C₁-C₅ alkyl. R² may be substituted or unsubstituted C₁-C₃ alkyl. R² may be substituted C₁-C₃ alkyl. R² may be substituted or unsubstituted alkyl. R² may be unsubstituted C₁-C₃ alkyl. R² may be methyl. R² may be ethyl.

R² may be hydrogen, halogen, —CN, —SH, —OH, —COOH, —NH₂, —CONH₂, —NO₂, —CF₃, —CCl₃, R^{2A}-substituted or unsubstituted alkyl or R^{2A}-substituted or unsubstituted heteroalkyl. R² may be hydrogen, halogen, —CN, —SR^{2A}, —OR^{2A}, —COOR^{2A}, —NR^{2A}R^{2B}, —CONR^{2A}R^{2B}, —NO₂, —CF₃, —CCl₃, R^{2A}-substituted or unsubstituted alkyl or R^{2A}-substituted or unsubstituted heteroalkyl. R² may be R^{2A}-substituted or unsubstituted alkyl. R² may be R^{2A}-substituted or unsubstituted heteroalkyl. R² may be hydrogen, R^{2A}-substituted or unsubstituted alkyl, or R^{2A}-substituted or unsubstituted heteroalkyl. R² may be hydrogen or R^{2A}-substituted or unsubstituted alkyl. R² may be hydrogen or R^{2A}-substituted alkyl. R² may be hydrogen or R^{2A}-substituted or unsubstituted C₁-C₅ alkyl. R² may be hydrogen or R^{2A}-substituted C₁-C₅ alkyl. R² may be hydrogen or R^{2A}-substituted or unsubstituted C₁-C₃ alkyl. R² may be hydrogen or R^{2A}-substituted C₁-C₃ alkyl. R² may be OR^{2A} or NR^{2A}R^{2B}, where R^{2A} and R^{2B} are independently hydrogen or substituted or unsubstituted alkyl. R² may be OR^{2A} or NR^{2A}R^{2B}, where R^{2A} and R^{2B} are independently hydrogen or methyl.

R² may be R^{2A}-substituted or unsubstituted alkyl. R² may be R^{2A}-substituted alkyl. R² may be R^{2A}-substituted or unsubstituted C₁-C₅ alkyl. R² may be R^{2A}-substituted C₁-C₅ alkyl. R² may be R^{2A}-substituted or unsubstituted C₁-C₃ alkyl. R² may be R^{2A}-substituted C₁-C₃ alkyl. R² may be hydrogen, halogen, —CF₃, or substituted or unsubstituted alkyl. R² may be —CF₃. R² may be hydrogen, halogen, —CF₃, or R^{2A}-substituted or unsubstituted alkyl.

R² may be R^{2A}-substituted or unsubstituted cycloalkyl (e.g. C₃-C₆ cycloalkyl), R^{2A}-substituted or unsubstituted heterocycloalkyl (e.g. 3 to 6 membered heterocycloalkyl), R^{2A}-substituted or unsubstituted aryl (e.g. phenyl), or R^{2A}-substituted or unsubstituted heteroaryl (e.g. 5 or 6 membered heteroaryl). R² may be hydrogen, halogen, —CN, —SH, —OH, —COOH, —NH₂, —CONH₂, —NO₂, —CF₃, —CCl₃, R^{2A}-substituted or unsubstituted alkyl or R^{2A}-substituted or unsubstituted heteroalkyl, R^{2A}-substituted or unsubstituted cycloalkyl (e.g. C₃-C₆ cycloalkyl), R^{2A}-substituted or unsubstituted heterocycloalkyl (e.g. 3 to 6 membered heterocycloalkyl), R^{2A}-substituted or unsubstituted aryl (e.g. phenyl), or R^{2A}-substituted or unsubstituted heteroaryl (e.g. 5 or 6 membered heteroaryl).

R^{2A} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, R^{2C}-substituted or unsubstituted alkyl, R^{2C}-substituted or unsubstituted heteroalkyl, R^{2C}-substituted or unsubstituted cycloalkyl, R^{2C}-substituted or unsubstituted heterocycloalkyl, R^{2C}-substituted unsubstituted aryl, or R^{2C}-substituted or unsubstituted heteroaryl. R^{2A} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl, unsubstituted heteroalkyl, unsubstituted cycloalkyl, unsubstituted heterocycloalkyl, unsubstituted aryl, or unsubstituted heteroaryl. R^{2A} may independently be halogen, —CF₃, —OH, —NH₂, unsubstituted alkyl, unsubstituted heteroalkyl, unsubstituted cycloalkyl, unsubstituted heterocycloalkyl, unsubstituted aryl, or unsubstituted heteroaryl. R^{2A} may independently be unsubstituted alkyl (e.g. C₁-C₃ alkyl), unsubstituted (e.g. 2 to 5 membered heteroalkyl, unsubstituted cycloalkyl (e.g. C₃-C₆ cycloalkyl), unsubstituted heterocycloalkyl (e.g. 3 to 6 membered heterocycloalkyl, unsubstituted aryl (e.g. phenyl), or unsubstituted heteroaryl (e.g. 5 or 6 membered heteroaryl).

R^{2B} and R^{2C} are independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R³ may be hydrogen, halogen, —CN, —SH, —OH, —COOH, —NH₂, —CONH₂, —NO₂, —CF₃₅—CCl₃, substituted or unsubstituted alkyl, or substituted or unsubstituted heteroalkyl. R³ may be hydrogen, halogen, —CF₃, or substituted or unsubstituted alkyl. R³ may be hydrogen, halogen or substituted or unsubstituted alkyl. R³ may be hydrogen, halogen or substituted alkyl. R³ may be hydrogen, halogen or unsubstituted alkyl. R³ may be hydrogen, halogen or substituted or unsubstituted C₁-C₅ alkyl. R³ may be hydrogen, halogen or substituted C₁-C₅ alkyl. R³ may be hydrogen, halogen or unsubstituted C₁-C₅ alkyl. R³ may be hydrogen, halogen or substituted or unsubstituted C₁-C₃ alkyl. R³ may be hydrogen, halogen or substituted C₁-C₃ alkyl. R³ may be hydrogen, halogen or unsubstituted C₁-C₃ alkyl.

R³ may be hydrogen, halogen, —CN, —SH, —OH, —COOH, —NH₂, —CONH₂, —NO₂, —CF₃, —CCl₃, R^{3A}-substituted or unsubstituted alkyl, or R^{3A}-substituted or unsubstituted heteroalkyl. R³ may be hydrogen, halogen, —CN, —SR^{3A}, —OR^{3A}, —COOR^{3A}, —NR^{3A}R^{3B}, —CONR^{3A}R^{3B}, —NO₂, —CF₃, —CCl₃, R^{3A}-substituted or unsubstituted alkyl, or R^{3A}-substituted or unsubstituted heteroalkyl. R³ may be hydrogen, halogen, —CF₃, or R^{3A}-substituted or unsubstituted alkyl. R³ may be hydrogen, halogen or R^{3A}-substituted or unsubstituted alkyl. R³ may be hydrogen, halogen or R^{3A}-substituted or unsubstituted C₁-C₅ alkyl. R³ may be hydrogen, halogen or R^{3A}-substituted C₁-C₅ alkyl. R³ may be hydrogen, halogen or R^{3A}-substituted or unsubstituted C₁-C₃ alkyl. R³ may be hydrogen, halogen or R^{3A}-substituted C₁-C₃ alkyl. R³ may be hydrogen. R³ may be hydrogen or halogen. R³ may be halogen. R³ may be —Cl. R³ may be —Br. R³ may be —I. R³ may be —F. R³ may be hydrogen or substituted or unsubstituted alkyl.

R³ may be substituted or unsubstituted alkyl. R³ may be substituted alkyl. R³ may be unsubstituted alkyl. R³ may be substituted or unsubstituted C₁-C₅ alkyl. R³ may be substituted C₁-C₅ alkyl. R³ may be unsubstituted C₁-C₅ alkyl. R³ may be substituted or unsubstituted C₁-C₃ alkyl. R³ may be substituted C₁-C₃ alkyl. R³ may be unsubstituted C₁-C₃ alkyl. R³ may be methyl. R³ may be ethyl. R³ may be propyl. R³ may be R^{3A}-substituted or unsubstituted alkyl. R³ may be R^{3A}-substituted alkyl. R³ may be R^{3A}-substituted or unsubstituted C₁-C₅ alkyl. R³ may be R^{3A}-substituted C₁-C₅ alkyl. R³ may be R^{3A}-substituted or unsubstituted C₁-C₃ alkyl. R³ may be R^{3A}-substituted C₁-C₃ alkyl.

R³ may be R^{3A}-substituted or unsubstituted alkyl (e.g. C₁-C₅ alkyl), R^{3A}-substituted or unsubstituted heteroalkyl (e.g. 2 to 6 membered heteroalkyl, R^{3A}-substituted or unsubstituted cycloalkyl (e.g. C₃-C₆ cycloalkyl), R^{3A}-substituted or unsubstituted heterocycloalkyl (e.g. 3 to 6 membered heterocycloalkyl), R^{3A}-substituted or unsubstituted aryl (e.g. phenyl), or R^{3A}-substituted or unsubstituted heteroaryl (e.g. 5 or 6 membered heteroaryl). R³ may be R^{3A}-substituted or unsubstituted cycloalkyl (e.g. C₃-C₆ cycloalkyl), R^{3A}-substituted or unsubstituted heterocycloalkyl (e.g. 3 to 6 membered heterocycloalkyl), R^{3A}-substituted or unsubstituted aryl (e.g. phenyl), or R^{3A}-substituted or unsubstituted heteroaryl (e.g. 5 or 6 membered heteroaryl). R³ may be hydrogen, halogen, —CN, —SH, —OH, —COOH, —NH₂, —CONH₂, —NO₂, —CF₃, —CCl₃, R^{3A}-substituted or unsubstituted alkyl or R^{3A}-substituted or unsubstituted heteroalkyl, R^{3A}-substituted or unsubstituted cycloalkyl (e.g. C₃-C₆ cycloalkyl), R^{3A}-substituted or unsubstituted heterocycloalkyl (e.g. 3 to 6 membered heterocycloalkyl), R^{3A}-substituted or unsubstituted aryl (e.g. phenyl), or R^{3A}-substituted or unsubstituted heteroaryl (e.g. 5 or 6 membered heteroaryl). R³ may be —OR^{3A} or —NR^{3A}R^{3B}, where R^{3A} and R^{3B} are independently hydrogen or substituted or unsubstituted alkyl. R³ may be —OR^{3A} or —NR^{3A}R^{3B}, where R^{3A} and R^{3B} are independently hydrogen or methyl.

R^{3A} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, R^{3C}-substituted or unsubstituted alkyl, R^{3C}-substituted or unsubstituted heteroalkyl, R^{1C}-substituted or unsubstituted cycloalkyl, R^{3C}-substituted or unsubstituted heterocycloalkyl, R^{3C}-substituted or unsubstituted aryl, or R^{3C}-substituted or unsubstituted heteroaryl. R³ may be substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl).

R^{3A} may be independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, substituted or unsubstituted alkyl, unsubstituted heteroalkyl, unsubstituted cycloalkyl, unsubstituted heterocycloalkyl, unsubstituted aryl, or unsubstituted heteroaryl. R^{3A} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl, unsubstituted heteroalkyl, unsubstituted cycloalkyl, unsubstituted heterocycloalkyl, unsubstituted aryl, or unsubstituted heteroaryl. R^{3A} may independently be halogen, —CF₃, —OH, —NH₂, unsubstituted alkyl, unsubstituted heteroalkyl, unsubstituted cycloalkyl, unsubstituted heterocycloalkyl, unsubstituted aryl, or unsubstituted heteroaryl. R^{3A} may independently be unsubstituted alkyl (e.g. C₁-C₃ alkyl), unsubstituted (e.g. 2 to 5 membered heteroalkyl, unsubstituted cycloalkyl (e.g. C₃-C₆ cycloalkyl), unsubstituted heterocycloalkyl (e.g. 3 to 6 membered heterocycloalkyl, unsubstituted aryl (e.g. phenyl), or unsubstituted heteroaryl (e.g. 5 or 6 membered heteroaryl).

R^{3B} and R^{3C} are independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R³ and R⁴ may be optionally joined together to form a substituted or unsubstituted cycloalkyl, substituted or unsubstituted heterocycloalkyl, substituted or unsubstituted aryl or substituted or unsubstituted heteroaryl. R³ and R⁴ may be optionally joined together to form a substituted or unsubstituted aryl or substituted or unsubstituted heteroaryl. R³ and R⁴ may be optionally joined together to form a substituted or unsubstituted aryl (e.g. phenyl). R³ and R⁴ may be optionally joined together to form a R^{3A}-substituted or unsubstituted aryl (e.g. phenyl). R³ and R⁴ may be joined together to form a substituted or unsubstituted 2-naptholyl. R³ and R⁴ may be joined together to form a R^{3A}-substituted or unsubstituted 2-naptholyl.

R⁴ may be hydrogen, halogen, —CN, —SH, —OH, —COOH, —NH₂, —CONH₂, —NO₂, —CF₃, —CCl₃, substituted or unsubstituted alkyl, or substituted or unsubstituted heteroalkyl. R⁴ may be hydrogen, halogen, —CN, —SH, —OH, —COOH, —NH₂, —CONH₂, —NO₂, —CF₃, —CCl₃, R^{4A}-substituted or unsubstituted alkyl, or R^{4A}-substituted or unsubstituted heteroalkyl. R⁴ may be hydrogen, halogen, —CN, —SR^{4A}, —OR^{4A}, —COOR^{4A}, —NR^{4A}R^{4B}, —CONR^{4A}R^{4B}, —NO₂, —CF₃, —CCl₃, R^{4A}-substituted or unsubstituted alkyl, or R^{4A}-substituted or unsubstituted heteroalkyl. R⁴ may be hydrogen or substituted or unsubstituted alkyl. R⁴ may be hydrogen or substituted alkyl. R⁴ may be hydrogen or unsubstituted alkyl. R⁴ may be hydrogen or substituted or unsubstituted C₁-C₅ alkyl. R⁴ may be hydrogen or substituted C₁-C₅ alkyl. R⁴ may be hydrogen or unsubstituted C₁-C₅ alkyl. R⁴ may be hydrogen or substituted or unsubstituted C₁-C₃ alkyl. R⁴ may be hydrogen or substituted C₁-C₃ alkyl. R⁴ may be hydrogen or unsubstituted C₁-C₃ alkyl. R⁴ may be hydrogen or methyl. R⁴ may be hydrogen or ethyl. R⁴ may be hydrogen. R⁴ may be halogen. R⁴ may be —Cl. R⁴ may be —Br. R⁴ may be —I. R⁴ may be —F. R⁴ may be —OR^{4A} or —NR^{4A}R^{4B}, where R^{4A} and R^{4B} are independently hydrogen or substituted or unsubstituted alkyl. R⁴ may be —OR^{4A} or —NR^{4A}R^{4B}, where R^{4A} and R^{4B} are independently hydrogen or methyl.

R⁴ may be substituted or unsubstituted alkyl. R⁴ may be substituted alkyl. R⁴ may be unsubstituted alkyl. R⁴ may be substituted or unsubstituted C₁-C₅ alkyl. R⁴ may be substituted C₁-C₅ alkyl. R⁴ may be unsubstituted C₁-C₅ alkyl. R⁴ may be substituted or unsubstituted C₁-C₃ alkyl. R⁴ may be substituted C₁-C₃ alkyl. R⁴ may be unsubstituted C₁-C₃ alkyl. R⁴ may be methyl. R⁴ may be ethyl. R⁴ may be propyl. R⁴ may be R^{4A}-substituted or unsubstituted alkyl. R⁴ may be R^{4A}-substituted alkyl. R⁴ may be R^{4A}-substituted or unsubstituted C₁-C₅ alkyl. R⁴ may be R^{4A}-substituted C₁-C₅ alkyl. R⁴ may be R^{4A}-substituted or unsubstituted C₁-C₃ alkyl. R⁴ may be R^{4A}-substituted C₁-C₃ alkyl.

R⁴ and R⁵ may be optionally joined together to form a substituted or unsubstituted cycloalkyl, substituted or unsubstituted heterocycloalkyl, substituted or unsubstituted aryl or substituted or unsubstituted heteroaryl. R⁴ and R⁵ may be optionally joined together to form a substituted or unsubstituted aryl or substituted or unsubstituted heteroaryl. R⁴ and R⁵ may be optionally joined together to form a substituted or unsubstituted aryl (e.g. phenyl). R⁴ and R⁵ may be optionally joined together to form a R^{4A}-substituted or unsubstituted aryl (e.g. phenyl).

R⁴ may be substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl).

R⁴ may be R^{4A}-substituted or unsubstituted alkyl (e.g. C₁-C₅ alkyl), R^{4A}-substituted or unsubstituted heteroalkyl (e.g. 2 to 6 membered heteroalkyl, R^{4A}-substituted or unsubstituted cycloalkyl (e.g. C₃-C₆ cycloalkyl), R^{4A}-substituted or unsubstituted heterocycloalkyl (e.g. 3 to 6 membered heterocycloalkyl), R^{4A}-substituted or unsubstituted aryl (e.g. phenyl), or R^{4A}-substituted or unsubstituted heteroaryl (e.g. 5 or 6 membered heteroaryl). R⁴ may be R^{4A}-substituted or unsubstituted cycloalkyl (e.g. C₃-C₆ cycloalkyl), R^{4A}-substituted or unsubstituted heterocycloalkyl (e.g. 3 to 6 membered heterocycloalkyl), R^{4A}-substituted or unsubstituted aryl (e.g. phenyl), or R^{4A}-substituted or unsubstituted heteroaryl (e.g. 5 or 6 membered heteroaryl). R⁴ may be hydrogen, halogen, —CN, —SH, —OH, —COOH, —NH₂, —CONH₂, —NO₂, —CF₃, —CCl₃, R^{4A}-substituted or unsubstituted alkyl or R^{4A}-substituted or unsubstituted heteroalkyl, R^{4A}-substituted or unsubstituted cycloalkyl (e.g. C₃-C₆ cycloalkyl), R^{4A}-substituted or unsubstituted heterocycloalkyl (e.g. 3 to 6 membered heterocycloalkyl), R^{4A}-substituted or unsubstituted aryl (e.g. phenyl), or R^{4A}-substituted or unsubstituted heteroaryl (e.g. 5 or 6 membered heteroaryl).

R^{4A} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, R^{4C}-substituted or unsubstituted alkyl, R^{4C}-substituted or unsubstituted heteroalkyl, R^{4C}-substituted or unsubstituted cycloalkyl, R^{4C}-substituted or unsubstituted heterocycloalkyl, R^{4C}-substituted or unsubstituted aryl, or R^{4C}-substituted or unsubstituted heteroaryl. R^{4A} may be independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, substituted or unsubstituted alkyl, unsubstituted heteroalkyl, unsubstituted cycloalkyl, unsubstituted heterocycloalkyl, unsubstituted aryl, or unsubstituted heteroaryl. R^{4A} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl, unsubstituted heteroalkyl, unsubstituted cycloalkyl, unsubstituted heterocycloalkyl, unsubstituted aryl, or unsubstituted heteroaryl. R^{4A} may independently be halogen, —CF₃, —OH, —NH₂, unsubstituted alkyl, unsubstituted heteroalkyl, unsubstituted cycloalkyl, unsubstituted heterocycloalkyl, unsubstituted aryl, or unsubstituted heteroaryl. R^{4A} may independently be unsubstituted alkyl (e.g. C₁-C₃ alkyl), unsubstituted (e.g. 2 to 5 membered heteroalkyl, unsubstituted cycloalkyl (e.g. C₃-C₆ cycloalkyl), unsubstituted heterocycloalkyl (e.g. 3 to 6 membered heterocycloalkyl, unsubstituted aryl (e.g. phenyl), or unsubstituted heteroaryl (e.g. 5 or 6 membered heteroaryl).

R^{4B} and R^{4C} are independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R⁵ may be hydrogen, halogen, —CN, —SH, —OH, —COOH, —NH₂, —CONH₂, —NO₂, —CF₃, —CCl₃, substituted or unsubstituted alkyl, or substituted or unsubstituted heteroalkyl. R⁵ may be hydrogen, halogen, —CN, —SH, —OH, —COOH, —NH₂, —CONH₂, —NO₂, —CF₃, —CCl₃, R^{5A}-substituted or unsubstituted alkyl, or R^{5A}-substituted or unsubstituted heteroalkyl. R⁵ may be hydrogen, halogen, —CN, —SR^{5A}, —OR^{5A}, —COOR^{5A}—NR^{5A}R^{5B}, —CONR^{5A}R^{5B}, —CF₃, —CCl₃, R^{5A}-substituted or unsubstituted alkyl, or R^{5A}-substituted or unsubstituted heteroalkyl. R⁵ may be hydrogen or substituted or unsubstituted alkyl. R⁵ may be hydrogen or substituted alkyl. R⁵ may be hydrogen or unsubstituted alkyl. R⁵ may be hydrogen or substituted or unsubstituted C₁-C₅ alkyl. R⁵ may be hydrogen or substituted C₁-C₅ alkyl. R⁵ may be hydrogen or unsubstituted C₁-C₅ alkyl. R⁵ may be hydrogen or substituted or unsubstituted C₁-C₃ alkyl. R⁵ may be hydrogen or substituted C₁-C₃ alkyl. R⁵ may be hydrogen or unsubstituted C₁-C₃ alkyl. R⁵ may be hydrogen or methyl. R⁵ may be hydrogen or ethyl.

R⁵ may be hydrogen, halogen, —CN, —SH, —OH, —COOH, —NH₂, —CONH₂, —NO₂, —CF₃, —CCl₃, R^{5A}-substituted or unsubstituted alkyl, or R^{5A}-substituted or unsubstituted heteroalkyl. R⁵ may be hydrogen or R^{5A}-substituted or unsubstituted alkyl. R⁵ may be hydrogen or R^{5A}-substituted alkyl. R⁵ may be hydrogen or R^{5A}-substituted or unsubstituted C₁-C₅ alkyl. R⁵ may be hydrogen or R^{5A}-substituted C₁-C₅ alkyl. R⁵ may be hydrogen or R^{5A}-substituted or unsubstituted C₁-C₃ alkyl. R⁵ may be hydrogen or R^{5A}-substituted C₁-C₃ alkyl. R⁵ may be hydrogen. R⁵ may be halogen. R⁵ may be —Cl. R⁵ may be —Br. R⁵ may be —I. R⁵ may be —F. R⁵ may be —OR^{5A} or —NR^{5A}R^{5B}, where R^{5A} and R^{5B} are independently hydrogen or substituted or unsubstituted alkyl. R⁵ may be —OR^{5A} or —NR^{5A}R^{5B}, where R^{5A} and R^{5B} are independently hydrogen or methyl.

R⁵ may be substituted or unsubstituted alkyl. R⁵ may be substituted alkyl. R⁵ may be unsubstituted alkyl. R⁵ may be substituted or unsubstituted C₁-C₅ alkyl. R⁵ may be substituted C₁-C₅ alkyl. R⁵ may be unsubstituted C₁-C₅ alkyl. R⁵ may be substituted or unsubstituted C₁-C₃ alkyl. R⁵ may be substituted C₁-C₃ alkyl. R⁵ may be unsubstituted C₁-C₃ alkyl. R⁵ may be methyl. R⁵ may be ethyl. R⁵ may be propyl. R⁵ may be R^{5A}-substituted or unsubstituted alkyl. R⁵ may be R^{5A}-substituted alkyl. R⁵ may be R^{5A}-substituted or unsubstituted C₁-C₅ alkyl. R⁵ may be R^{5A}-substituted C₁-C₅ alkyl. R⁵ may be R^{5A}-substituted or unsubstituted C₁-C₃ alkyl. R⁵ may be R^{5A}-substituted C₁-C₃ alkyl.

R⁵ may be hydrogen, halogen, —CF₃, or substituted or unsubstituted alkyl. R⁵ may be hydrogen, halogen, —CF₃, or substituted or unsubstituted alkyl, or substituted or unsubstituted aryl. R⁵ may be phenol. R⁵ may be 2-naphthol. R⁴ and R⁵ may be joined together to form a substituted or unsubstituted 2-naptholyl. R⁴ and R⁵ may be joined together to form a R^{4A}-substituted or unsubstituted 2-naptholyl.

R⁵ may be R^{5A}-substituted or unsubstituted alkyl (e.g. C₁-C₅ alkyl), R^{5A}-substituted or unsubstituted heteroalkyl (e.g. 2 to 6 membered heteroalkyl, R^{5A}-substituted or unsubstituted cycloalkyl (e.g. C₃-C₆ cycloalkyl), R^{5A}-substituted or unsubstituted heterocycloalkyl (e.g. 3 to 6 membered heterocycloalkyl), R^{5A}-substituted or unsubstituted aryl (e.g. phenyl), or R^{5A}-substituted or unsubstituted heteroaryl (e.g. 5 or 6 membered heteroaryl). R⁴ may be R^{5A}-substituted or unsubstituted cycloalkyl (e.g. C₃-C₆ cycloalkyl), R^{5A}-substituted or unsubstituted heterocycloalkyl (e.g. 3 to 6 membered heterocycloalkyl), R^{5A}-substituted or unsubstituted aryl (e.g. phenyl), or R^{5A}-substituted or unsubstituted heteroaryl (e.g. 5 or 6 membered heteroaryl). R⁵ may be hydrogen, halogen, —CN, —SH, —OH, —COOH, —NH₂, —CONH₂, —NO₂, —CF₃, —CCl₃, R^{5A}-substituted or unsubstituted alkyl or R^{5A}-substituted or unsubstituted heteroalkyl, R^{5A}-substituted or unsubstituted cycloalkyl (e.g. C₃-C₆ cycloalkyl), R^{5A}-substituted or unsubstituted heterocycloalkyl (e.g. 3 to 6 membered heterocycloalkyl), R^{5A}-substituted or unsubstituted aryl (e.g. phenyl), or R^{5A}-substituted or unsubstituted heteroaryl (e.g. 5 or 6 membered heteroaryl).

R^{5A} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl. R^{5A} may be independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. R^{5A} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl, unsubstituted heteroalkyl, unsubstituted cycloalkyl, unsubstituted heterocycloalkyl, unsubstituted aryl, or unsubstituted heteroaryl. R^{5A} may independently be halogen, —CF₃, —OH, —NH₂, unsubstituted alkyl, unsubstituted heteroalkyl, unsubstituted cycloalkyl, unsubstituted heterocycloalkyl, unsubstituted aryl, or unsubstituted heteroaryl. R^{5A} may independently be unsubstituted alkyl (e.g. C₁-C₃ alkyl), unsubstituted (e.g. 2 to 5 membered heteroalkyl, unsubstituted cycloalkyl (e.g. C₃-C₆ cycloalkyl), unsubstituted heterocycloalkyl (e.g. 3 to 6 membered heterocycloalkyl, unsubstituted aryl (e.g. phenyl), or unsubstituted heteroaryl (e.g. 5 or 6 membered heteroaryl).

R^{5B} and R^{5C} are independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R², R³, R⁴, and R⁵ may independently be hydrogen, halogen, —CF₃, or substituted or unsubstituted alkyl.

R⁶ may be —OR⁹ or —N(R⁸)(R⁹). R⁶ may be —OR⁹. R⁶ may be —OR⁹, where R⁹ is hydrogen. R⁶ may be —OR⁹, where R⁹ is substituted or unsubstituted alkyl. R⁶ may be —OR⁹, where R⁹ is R^{9A}-substituted or unsubstituted alkyl. R⁶ may be —OR⁹, where R⁹ is hydrogen or R^{9A}-substituted or unsubstituted alkyl. R⁶ may be —OR⁹, where R⁹ is a hydrophilic polysaccharide. R⁶ may be —N(R⁸)(R⁹). R⁶ may be —N(R⁸)(R⁹), where R⁸ is hydrogen. R⁶ may be —N(R⁸)(R⁹), where R⁸ is substituted or unsubstituted alkyl. R⁶ may be —N(R⁸)(R⁹), where R⁹ is hydrogen. R⁶ may be —N(R⁸)(R⁹), where R⁹ is substituted or unsubstituted alkyl. R⁶ may be —N(R⁸)(R⁹), where R⁹ is a hydrophilic polysaccharide. R⁶ may be —N(R⁸)(R⁹), where R⁸ and R⁹ are hydrogen. R⁶ may be —N(R⁸)(R⁹), where R⁸ is hydrogen and R⁹ is substituted or unsubstituted alkyl. R⁶ may be —N(R⁸)(R⁹), where R⁸ is hydrogen and R⁹ is R^{9A}-substituted or unsubstituted alkyl.

R⁶ may be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ may be R^{6A}-substituted or unsubstituted alkyl, R^{6A}-substituted or unsubstituted heteroalkyl, R^{6A}-substituted or unsubstituted cycloalkyl, R^{6A}-substituted or unsubstituted heterocycloalkyl, R^{6A}-substituted or unsubstituted aryl, or R^{6A}-substituted or unsubstituted heteroaryl. R⁶ may be —OH, —N(R⁸)(R⁹), substituted or unsubstituted alkyl, substituted or unsubstituted heteroalkyl. R⁶ may be substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl).

R⁸ may independently be hydrogen. R⁸ may independently be substituted or unsubstituted alkyl. R⁸ may independently be substituted alkyl. R⁸ may independently be unsubstituted alkyl. R⁸ may independently be substituted or unsubstituted C₁-C₅ alkyl. R⁸ may independently be substituted C₁-C₅ alkyl. R⁸ may independently be unsubstituted C₁-C₅ alkyl. R⁸ may independently be R^{8A}-substituted or unsubstituted alkyl. R⁸ may independently be R^{8A}-substituted alkyl. R⁸ may independently be R^{8A}-substituted or unsubstituted C₁-C₅ alkyl. R⁸ may independently be R^{8A}-substituted C₁-C₅ alkyl.

R⁹ may independently be hydrogen. R⁹ may independently be substituted or unsubstituted alkyl. R⁹ may independently be a hydrophilic polysaccharide. R⁹ may independently be substituted or unsubstituted alkyl. R⁹ may independently be substituted alkyl. R⁹ may independently be unsubstituted alkyl. R⁹ may independently be substituted or unsubstituted C₁-C₅ alkyl. R⁹ may independently be substituted C₁-C₅ alkyl. R⁹ may independently be unsubstituted C₁-C₅ alkyl. R⁹ may independently be R^{9A}-substituted or unsubstituted alkyl. R⁹ may independently be R^{9A}-substituted alkyl. R⁹ may independently be R^{9A}-substituted or unsubstituted C₁-C₅ alkyl. R⁹ may independently be R^{9A}-substituted C₁-C₅ alkyl.

R^{6A} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, R^{6C}-substituted or unsubstituted alkyl, R^{6C}-substituted or unsubstituted heteroalkyl, R^{6C}-substituted or unsubstituted cycloalkyl, R^{6C}-substituted or unsubstituted heterocycloalkyl, R^{6C}-substituted or unsubstituted aryl, or R^{6C}-substituted or unsubstituted heteroaryl. R^{6A} may be independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. R^{6A} may independently be hydrogen, halogen, —CF₃, —OH, —NH₂, —N(CH₃)₂, —C(O)H, —C(O)CH₃, —C(O)OH, —C(O)OCH₃, —CONH₂, —NO₂, unsubstituted alkyl, unsubstituted heteroalkyl, unsubstituted cycloalkyl, unsubstituted heterocycloalkyl, unsubstituted aryl, or unsubstituted heteroaryl. R^{6A} may independently be halogen, —CF₃, —OH, —NH₂, unsubstituted alkyl, unsubstituted heteroalkyl, unsubstituted cycloalkyl, unsubstituted heterocycloalkyl, unsubstituted aryl, or unsubstituted heteroaryl. R^{6A} may independently be unsubstituted alkyl (e.g. C₁-C₃ alkyl), unsubstituted (e.g. 2 to 5 membered heteroalkyl, unsubstituted cycloalkyl (e.g. C₃-C₆ cycloalkyl), unsubstituted heterocycloalkyl (e.g. 3 to 6 membered heterocycloalkyl, unsubstituted aryl (e.g. phenyl), or unsubstituted heteroaryl (e.g. 5 or 6 membered heteroaryl).

R^{6C} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

R⁷ may independently be halogen, —CN, —SH, —OH, —COOH, —NH₂, —CONH₂, —NO₂, —CF₃, —CCl₃, -L¹-R¹³, substituted or unsubstituted alkyl, or substituted or unsubstituted heteroalkyl. R⁷ may independently be halogen, —CN, —SH, —OH, —COOH, —NH₂, —CONH₂, —NO₂, —CF₃, —CCl₃. The symbol z1 may be 0 (i.e. R⁷ is absent such that phenyl moiety is unsubstituted (i.e. valency filled with hydrogen)). R⁷ may independently be substituted or unsubstituted alkyl (e.g. C₁-C₅ alkyl), substituted or unsubstituted heteroalkyl (e.g. 2 to 6 membered heteroalkyl), substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl). R⁷ may independently be substituted or unsubstituted phenyl. R⁷ may independently be -L¹-R¹³.

R⁷ may independently be R¹⁰-substituted or unsubstituted alkyl (e.g. C₁-C₅ alkyl), R¹⁰-substituted or unsubstituted heteroalkyl (e.g. 2 to 6 membered heteroalkyl), R¹⁰-substituted or unsubstituted cycloalkyl (e.g. C₃-C₆ cycloalkyl), R¹⁰-substituted or unsubstituted heterocycloalkyl (e.g. 3 to 6 membered heterocycloalkyl), R¹⁰-substituted or unsubstituted aryl (e.g. phenyl), or R¹⁰-substituted or unsubstituted heteroaryl (e.g. 5 or 6 membered heteroaryl). R⁷ may independently be R¹⁰-substituted or unsubstituted alkyl (e.g. C₁-C₅ alkyl) or R¹⁰-substituted or unsubstituted heteroalkyl (e.g. 2 to 6 membered heteroalkyl). R⁷ may independently be R¹⁰-substituted or unsubstituted cycloalkyl (e.g. C₃-C₆ cycloalkyl), R¹⁰-substituted or unsubstituted heterocycloalkyl (e.g. 3 to 6 membered heterocycloalkyl), R¹⁰-substituted or unsubstituted aryl (e.g. phenyl), or R¹⁰-substituted or unsubstituted heteroaryl (e.g. 5 or 6 membered heteroaryl).

L¹ may be substituted or unsubstituted alkylene. L¹ may be substituted or unsubstituted heteroalkylene. L¹ may be —C(O)—X¹-L^{1A}-X²—C(O)—, where X¹ and X² are independently —O— or —NH—; and L^{1A} is substituted or unsubstituted alkylene or substituted or unsubstituted heteroalkylene. L^{1A} may be -L^{1B}-(CH₂CH₂O)ₙ— where the symbol n is an integer from 1 to 100, and L^{1B} is unsubstituted C₁-C₁₀ alkyl. The symbol n may be an integer from 1 to 10. L^{1B} is ethylene. The symbol n may be an integer from 1 to 10 where L^{iB} is ethylene. L¹ may be —C(O)O—CH₂CH₂—(OCH₂CH₂)ₙ—NH—C(O)—, where the symbol n is 1 to 10.

R¹³ may be substituted or unsubstituted cycloalkyl. R¹³ may be substituted or unsubstituted heterocycloalkyl. R¹³ may be substituted or unsubstituted aryl. R¹³ may be substituted or unsubstituted heteroaryl. R¹³ may be R¹⁰-substituted or unsubstituted aryl or R¹⁰-substituted or unsubstituted heteroaryl. R¹³ may be R¹⁰-substituted phenyl.

R¹⁰ is independently halogen, —CN, —SH, —OH, —COOH, —NH₂, —CONH₂, —NO₂, —CF₃, —CCl₃, R^{10A}-substituted or unsubstituted alkyl, R^{10A}-substituted or unsubstituted heteroalkyl, R^{10A}-substituted or unsubstituted cycloalkyl, R^{10A}-substituted or unsubstituted heterocycloalkyl, R^{10A}-substituted or unsubstituted aryl, or R^{10A}-substituted or unsubstituted heteroaryl. R¹° may independently be halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{10A} is independently halogen, —CN, —SH, —OH, —COOH, —NH₂, —CONH₂, —NO₂, —CF₃, —CCl₃, R^{10B}-substituted or unsubstituted alkyl, R^{10B}-substituted or unsubstituted heteroalkyl, R^{10B}-substituted or unsubstituted cycloalkyl, R^{10B}-substituted or unsubstituted heterocycloalkyl, R^{10B}-substituted or unsubstituted aryl, or R^{10B}-substituted or unsubstituted heteroaryl.

R^{10B} is independently hydrogen, oxo, halogen, —CCl₃, —CI₃, —CBr₃, —CF₃, —CN, —OH, —NH₂, —N(CH₃)₂, —C(O)H, —C(O)CH₃, —C(O)OH, —C(O)OCH₃, —CONH₂, —NO₂, —SH, —S(O)₂Cl, —S(O)₃ H, —S(O)₄H, —S(O)₂NH₂, —NHNH₂, —ONH₂, —NHOH, —NHC(O)NHNH₂, —NHC(O)NH₂, —NHS(O)₂H, —NHC(O)H, —NHC(O)—OH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

The compound of formula (I) may have the formula:

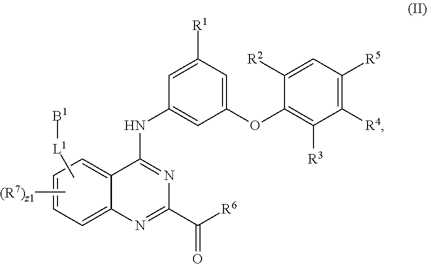

or a pharmaceutically acceptable salt thereof. The symbol z1 in formula (II) is 0 to 3. R¹, R², R³, R⁴, R⁵, R⁶, R⁷, L¹ and z1 are as described herein. B¹ is a purine base or analog thereof

The compound of formula (I) may have the formula:

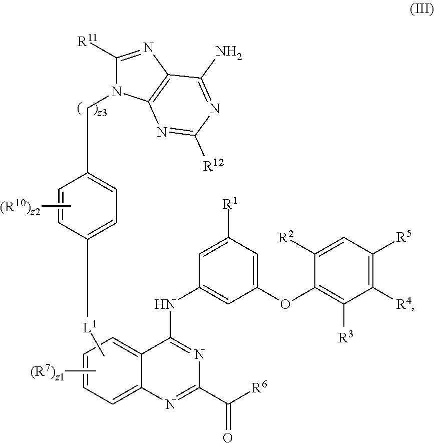

or a pharmaceutically acceptable salt thereof. The symbol z1 in formula (II) is 0 to 3. R¹, R², R³, R⁴, R⁵, R⁶, R⁷, R¹⁰, L¹ and z1 are as described herein. R¹¹ is —SR^{11A} or —OR^{11A}. R^{11A} is hydrogen, substituted or unsubstituted alkyl (e.g. C₁-C₅ alkyl), or substituted or unsubstituted heteroalkyl (e.g. 2 to 6 membered heteroalkyl), substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl). R¹² is hydrogen, halogen, —NO₂, —OH, —SH, —CN, —COOH, substituted or unsubstituted alkyl (e.g. C₁-C₅ alkyl), or substituted or unsubstituted heteroalkyl (e.g. 2 to 6 membered heteroalkyl), substituted or unsubstituted cycloalkyl (e.g. C₃-C₆ cycloalkyl), substituted or unsubstituted heterocycloalkyl (e.g. 3 to 6 membered heterocycloalkyl), substituted or unsubstituted aryl (e.g. phenyl), or substituted or unsubstituted heteroaryl (e.g. 5 or 6 membered heteroaryl). The symbol z2 is an integer from 0 to 4. The symbol z3 is an integer from 1 to 10.

The compound may be a compound set forth in Table 1.

The compound may have the formula:

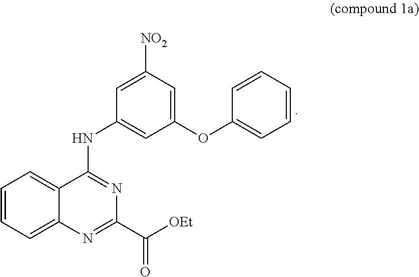 [SMILES: CCOC(=O)c1nc(Nc2cc(Oc3ccccc3)cc([N+](=O)[O-])c2)c2ccccc2n1]

### II. PHARMACEUTICAL COMPOSITIONS

Further provided herein are pharmaceutical compositions of the compounds described herein. In one aspect is a pharmaceutical composition including a pharmaceutically acceptable excipient and a compound described herein (e.g. a compound of formula (I) or as set forth in Table 1). The compound may be a compound as set forth in Table 1. The pharmaceutical composition may include a second agent in a therapeutically effective amount. The pharmaceutical composition may include a second agent where the second agent treats cancer. The second agent may be an anti-cancer agent as described herein. The pharmaceutical composition may include a second agent where the second agent treats an autoimmune disease. The pharmaceutical composition may include a second agent where the second agent treats inflammation. The second agent may be an anti-inflammatory agent as described herein. The pharmaceutical composition may include a second agent where the second agent treats an infectious disease (e.g. an anti-viral agent or an antibiotic). The terms “pharmaceutically acceptable” and the like refer, in the usual and customary sense, to a compound or composition which can be administered to a subject without causing a significant adverse toxicological effect, as judged by a medical or veterinary professional.

The pharmaceutical compositions include optical isomers, diastereomers, or pharmaceutically acceptable salts of the compounds described herein. The pharmaceutical compositions may include a compound of the present invention and citrate as a pharmaceutically acceptable salt. The compounds included in the pharmaceutical composition may be covalently attached to a carrier moiety. Alternatively, the compound included in the pharmaceutical composition is not covalently linked to a carrier moiety.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation, increase immune response) or with adjuvants when such formulation is directed to vaccine compositions. An example of co-administration when administered as a vaccine composition described herein is a prime-boost method of administration.

The pharmaceutical composition includes a compound described herein. The pharmaceutical composition may include a compound described herein. The pharmaceutical composition may include a compound described herein. The pharmaceutical composition may include a compound having a structure as set forth in Table 1.

The pharmaceutical composition may further include an antigen described herein. The pharmaceutical composition may include a TLR modulator. The pharmaceutical composition may include an adjuvant described herein.

A. Formulations

The compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention, i.e., “pharmaceutical formulation.”

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify. Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in PHARMACEUTICAL SCIENCES (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided by the present invention include compositions wherein the active ingredient is contained in a therapeutically effective amount. The actual amount effective for a particular application will depend, inter alia, on the condition being treated. For example, when administered in methods to treat cancer, such compositions will contain an amount of active ingredient effective to achieve the desired result (e.g. decreasing the number of cancer cells in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the invention.

For compounds described herein the therapeutically effective amount can be initially determined from cell culture assays. Target concentrations will be those concentrations of active compound(s) that are capable of eliciting innate immune response as measured, for example, using the methods described. Therapeutically effective amounts for use in humans may be determined from animal models. For example, a dose for humans can be formulated to achieve a concentration that has been found to be effective in animals. The dosage in humans can be adjusted by monitoring effectiveness and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. The dosage range may be 0.001% to 10% w/v. The dosage range may be 0.1% to 5% w/v. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

### III. VACCINE COMPOSITIONS

Further provided herein are vaccine compositions that include a compound a described herein. In one aspect is a vaccine composition that includes an antigen and a compound described herein. Vaccine compositions described herein may include an agent (e.g., antigen) that resembles a disease-causing microorganism, e.g., a killed or weakened form of the disease-causing microorganism, or a toxin, protein (e.g., surface protein) or other component of a disease-causing microorganism. The term “resembles” in this context means that the agent is sufficiently similar in structure to a disease-causing microorganism (e.g., bacteria, virus), or component thereof, such that the vaccine can improve immunity to a disease in a subject.

As well known in the art, vaccine compositions can include a suspending fluid (e.g., sterile water, saline, or fluids containing protein); excipients (e.g., preservatives and stabilizers), and adjuvants or enhancers (e.g., a compound described herein) that help improve the effectiveness of the vaccine. Vaccine compositions also may include small amounts of the culture material used to grow the virus or bacteria used in the vaccine, e.g., chicken egg protein and the like.

The terms “vaccine excipient” and the like refer to a pharmaceutically acceptable excipient used in the formulation of vaccines. As known in the art, vaccine excipient and adjuvants include, without limitation: 2-phenoxyethanol, acetone, albumin, alcohol, aluminum hydroxide, aluminum hydroxyphosphate sulfate, aluminum phosphate, aluminum potassium sulfate, aluminum potassium sulfate, amino acid supplement, amino acids, ammonium phosphate, ammonium sulfate, amorphous aluminum hydroxyphosphate sulfate, amphotericin B, anhydrous lactose, arginine, ascorbic acid, asparagine, benzethonium chloride, beta-propiolactone, beta-propiolactone, bovine albumin, bovine calf serum, bovine calf serum, bovine extract, bovine muscle tissue, bovine serum albumin, calcium carbonate, calcium chloride, calf serum, calf serum protein, carbohydrates, casamino acids, casein, castor oil, cell culture media, cellulose acetate phthalate, chick embryo cell culture, chicken protein, chlortetracycline, citric acid, CMRL 1969 medium (supplemented with calf serum), dextran, dextrose, D-fructose, dibasic potassium phosphate, dibasic sodium phosphate, disodium phosphate, D-mannose, Dulbecco's Modified Eagle Medium (DMEM) human serum albumin, Dulbecco's Modified Eagle's Medium, Eagle MEM modified medium, EDTA, egg protein, ethylene diamine tetraacetic acid (EDTA), FD&C Yellow #6 aluminum lake dye, Fenton medium (containing bovine extract), fetal bovine serum, formaldehyde, formalin, Franz complete medium, galactose, gelatin, gentamicin sulfate, glutamate, glutaraldehyde, glycerin, hemin chloride, hexadecyltrimethylammonium bromide, human albumin, human diploid cell cultures, human serum albumin, human-diploid fibroblast cell cultures (WI-38), hydrocortisone, hydrolyzed gelatin, hydrolyzed porcine gelatin, inorganic salts and sugars, insect cell and viral protein, iron ammonium citrate, lactalbumin hydrolysate, lactose, Latham medium derived from bovine casein, L-histidine, lipids, magnesium stearate, magnesium sulfate, mannitol, micro crystalline cellulose, mineral salts, modified Latham medium (derived from bovine casein), modified Mueller and Miller medium, modified Mueller's growth medium, modified Mueller's media (containing bovine extracts), modified Mueller-Miller casamino acid medium (without beef heart infusion), modified Mueller-Miller casamino acid medium without beef heart infusion, modified Stainer-Scholte liquid medium, monkey kidney cells, monobasic potassium phosphate, monobasic sodium phosphate, monosodium glutamate, monosodium L-glutamate, monosodium phosphate, MRC-5 (human diploid) cells, MRC-5 cells, MRC-5 cellular proteins, MRC-5 human diploid cells, Mueller and Miller medium, Mueller Hinton agar, Mueller's Growth Medium, Mueller-Miller casamino acid medium (without beef heart infusion), neomycin, neomycin sulfate, nicotinamide adenine dinucleotide, nonylphenol ethoxylate, octylphenol ethoxylate (Triton™ X-100), ovalbumin, peptone, phenol, phosphate, phosphate buffer, phosphate buffers, phosphate buffers, plasdone C, polacrilin potassium, polydimethylsiloxane, polygeline (processed bovine 14 gelatin), polymyxin, polymyxin B, polymyxin B sulfate, polysorbate 20, polysorbate 80, potassium aluminum sulfate, potassium chloride, potassium glutamate, potassium phosphate, potassium phosphate dibasic, potassium phosphate monobasic, protamine sulfate, recombinant human albumin, residual components of MRC-5 cells including DNA and protein, sodium bicarbonate, sodium borate, sodium citrate, sodium deoxycholate, sodium dihydrogen phosphate dehydrate, sodium hydroxide, sodium metabisulphite, sodium phosphate, sodium phosphate dibasic, sodium phosphate monobasic, sodium phosphate monobasic monohydrate, sodium taurodeoxycholate, sorbitol, soy peptone, Stainer-Scholte liquid medium, Stainer-Scholte medium, Stainer-Scholte medium (modified by the addition of casamino acids and dimethyl-beta-cyclodextrin), streptomycin, succinate buffer, sucrose, thimerosal, Vero (monkey kidney) cells, vitamins, Watson Scherp media, WI-38 human diploid lung fibroblasts, xanthan, yeast, yeast extract, yeast protein, α-tocopheryl hydrogen succinate, and β-propiolactone. An adjuvant is as described herein and may include compounds described herein.

The vaccine composition may include an antigen, a compound described herein, and at least one vaccine excipient as described herein. The vaccine composition may include an antigen, a compound described herein, and an adjuvant described herein. The vaccine composition may include an antigen, a compound described herein, and two or more adjuvants described herein. The vaccine composition may include an antigen, a compound described herein, a vaccine excipient described herein and an adjuvant described herein. The vaccine may include a TLR modulator.

The adjuvant or compound described herein in the vaccine composition may agonize or activate a Toll-like receptor. There are three general categories of Toll-like receptor (TLR) ligands: proteins, nucleic acids and lipid-based elements. As known in the art, TLRs recognize conserved structures of microbes and endogenous (host-derived) molecules. TLRs that recognize bacterial and fungal components may be localized on the cell surface, whereas TLRs that recognize viral or microbial nucleic acids are localized to intracellular membranes such as endosomes or phagosomes. Thus, in embodiments, different TLRs are amenable to targeting by different types of agents. Cell surface TLRs can be targeted by small molecules (for example, eritoran that inhibits TLR4) and antibodies (for example, OPN-305, which targets TLR2), whereas the intracellular nucleic-acid sensing TLRs may be responsive to targeting with modified oligonucleotides.

Specifically, TLR2 may be expressed on monocytes, mature macrophages and dendritic cells, and mast cells. TLR2 may specifically recognize components from Gram-positive bacteria, including lipoteichoic acid (LTA) with the assistance of the scavenger receptor CD36. TLR2 can form a heterodimer with TLR1 to recognize triacylated lipopeptides, such as the synthetic ligand Pam3CSK4, or with TLR6 to recognize diacylated lipopeptides like MALP-2. TLR1, TLR2 and TLR6 are highly similar and arose from an evolutionary gene duplication event. The dimerization of these TLRs allows the recognition of a more specific and wider array of microbial components. See e.g., Underhill, D., et al., Nature 1999, 401:811-815. It is reported that TLR2 and TLR4 are stimulated upon contact with P-MAPA (Protein aggregated Magnesium-Ammonium Phospholinoleate-Palmitoleate Anhydride, as known in the art. Reported TLR2 antagonists include OPN-305 and OPN-401. TLR3 is an endosomal TLR expressed in dendritic cells. It recognizes double stranded RNA, which may be produced by replicating viruses and the synthetic ligand polyriboinosinic polyribocytidylic acid (poly I:C). Reported TLR3 agonists include Rintatolimod. TLR4 recognizes lipopolysaccharide (LPS) from Gram-negative bacteria. The recognition process may be enhanced by LPS-binding protein (LBP), which carries LPS to the CD14 molecule, where it is then presented to the MD-2-TLR4 complex. TLR4 is expressed predominately on monocytes, mature macrophages and dendritic cells, mast cells and the intestinal epithelium. TLR modulators (antagonists) for TLR4 include NI-0101, 1A6, AV411, Eritoran, and TAK-242. TLR modulators (agonists) for TLR4 include Pollinex® Quattro.

TLR5 binds flagellin, a constituent of bacterial flagella. TLR5 is expressed primarily on cells of the intestinal epithelium and in monocytes, macrophages and dendritic cells. Reported TLR5 agonists include VAX-102. TLR7 and TLR8 are found in endosomes of monocytes and macrophages, with TLR7 also being expressed on plasmacytoid dendritic cells, and TLR8 also being expressed in mast cells. Both these receptors may recognize single stranded RNA from viruses. Synthetic ligands, such as R-848 and imiquimod, can be used to activate the TLR7 and TLR8 signaling pathways. TLR9 is expressed in endosomes of monocytes, macrophages and plasmacytoid dendritic cells, and may act as a receptor for unmethylated CpG islands found in bacterial and viral DNA. Synthetic oligonucleotides that contain unmethylated CpG motifs are used to activate TLR9. For example, class A oligonucleotides target plasmacytoid dendritic cells and strongly induce IFNα production and antigen presenting cell maturation, while indirectly activating natural killer cells. Class B oligonucleotides target B cells and natural killer cells and induce little interferon-α (IFNα). Class C oligonucleotides target plasmacytoid dendritic cells and are potent inducers of IFNα. This class of oligonucleotides is involved in the activation and maturation of antigen presenting cells, indirectly activates natural killer cells and directly stimulates B cells. Reported TLR modulators (agonist) for TLR7 include ANA772, Imiquimod, and AZD8848. TLR modulators (agonist) for TLR8 include VTX-1463. TLR modulators (agonist) for TLR7 and TLR8 include Resiquimod. TLR modulators (antagonists) for TLR7 and TLR9 include IRS-954, and IMO-3100. TLR9 agonists include SD-101, IMO-2125, Bio Thrax plus CpG-7909, AVE0675, QAX-935, SAR-21609, and DIMS0150.

Accordingly, the vaccine composition may include a TLR modulator, where the TLR modulator is an agonist of TLR2, TLR3, TLR4, TLR5, TLR7, TLR8 or TLR9. The vaccine composition may include a TLR modulator, where the TLR modulator is an antagonist of TLR2, TLR3, TLR4, TLR5, TLR7, TLR8 or TLR9. The vaccine composition may include a TLR modulator selected from TLR modulators for TLR2, TLR3, TLR4, TLR5, TLR7, TLR8 or TLR9. The vaccine composition may include a plurality of TLR modulators selected from TLR modulators for TLR2, TLR3, TLR4, TLR5, TLR7, TLR8 or TLR9.

The compound described may be present in the vaccine in an amount sufficient to synergistically increase the activity of the TLR modulator. The terms “synergistically,” “synergy”, “synergism,” “synergistic” and like refer to a measured effect of compounds administered in combination where the measured effect is greater than the sum of the individual effects of each of the compounds administered alone as a single agent. The vaccine composition may include a TLR modulator, wherein the TLR modulator is an agonist of TLR2, TLR3, TLR4, TLR5, TLR7, TLR8 or TLR9, and the vaccine activity is greater than the sum of the individual effects of each of i) a compound described herein, and ii) the TLR modulator, each administered without the other. The TLR modulator may be a TLR2 agonist. The TLR modulator may be a TLR3 agonist. The TLR modulator may be a TLR4 agonist. The TLR modulator may be a TLR5 agonist. The TLR modulator may be a TLR7 agonist. The TLR modulator may be a TLR8 agonist. The TLR modulator may be a TLR9 agonist. The TLR modulator may be a TLR2 antagonist. The TLR modulator may be a TLR3 antagonist. The TLR modulator may be a TLR4 antagonist. The TLR modulator may be a TLR5 antagonist. The TLR modulator may be a TLR7 antagonist. The TLR modulator may be a TLR8 antagonist. The TLR modulator may be a TLR9 antagonist.

Further provided herein are biological cells which include a compound described herein. The term “biological cell” is used in accordance with its usual and customary sense, and refers to the basic unit of living organisms, both unicellular and multicellular. The biological cell can be an isolated biological cell removed from the original host organism (e.g., in cell culture, explanted and the like), or a biological cell within the host organism (i.e., not removed from the host organism). Thus, the biological cell may form part of an organism. The organism is may be a mammal. The organism may be a human. The biological cell is a diseased biological cell (e.g. a cancer cell). Contacting a biological cell with a compound described herein may induce cytokine expression in the biological cell. The cytokine may be IL-6 or IL-8. The cell may be a dendritic cell (DC). Contacting the dendritic cell with a compound described herein may induce expression of CD40, CD80, or CD83. The expression may be higher than expression in a control cell (e.g. a cell not contacted with a compound described herein).

In another aspect, is a mixture that includes a TLR modulator and a compound described herein. The mixture may be contained within a single vessel or container. The mixture may be diluted in a liquid (e.g., a liquid pharmaceutically acceptable excipient) for administration to a subject. The mixture may be a pharmaceutical formulation described herein. The TLR modulator is as described herein and accordingly may be an antagonist or agonist of a TLR. The mixture may be a liquid mixture or a powder mixture. The mixture may be a liquid that is a pharmaceutically acceptable formulation. The mixture may be a powder that is a pharmaceutically acceptable formulation.

The compounds described herein may be present in the mixture in an amount sufficient to increase the TLR modulator activity in a biological cell. The terms “TLR activity” and the like refer to the biological activity of any of TLR2, TLR3, TLR4, TLR5, TLR7, TLR8 or TLR9. The term “increase the TLR modulator activity” means increasing the antagonist activity of a TLR modulator, or increasing the agonist activity of a TLR modulator, relative to the absence of the compound. Conversely, the term “decrease the TLR modulator activity” means decreasing the antagonist activity of a TLR modulator, or decreasing the agonist activity of a TLR modulator, relative to the absence of the compound. Accordingly, the compound may be present in an amount sufficient to agonize the TLR activity in a biological cell. The compound may be present in an amount sufficient to antagonize the TLR activity in a biological cell.

The compounds described herein may be present in the mixture in an amount sufficient to synergistically increase TLR agonist activity in a biological cell. The TLR may be one of TLR2, TLR3, TLR4, TLR5, TLR7, TLR8 or TLR9. The TLR modulator may be an agonist of TLR7. The observed increase in immunoglobulin or IFN-γ may be greater than the observed sum of the individual effects of i) a compound described herein and ii) the TLR modulator, when administered as single agents. In The TLR modulator may be an agonist of the TLR. The TLR modulator may be an agonist of TLR7. See Example 13. The mixture may agonize one or more TLRs.

The biological cell may be in an organism (e.g., a human). The biological cell may be in a human. TLR agonistic activity may be increased. The TLR4 receptor may be agonized. When a TLR is agonized, the innate immune may be activated to provide a rapid response to perceived threats before pathogen-specific adaptive immunity can be established. When a TLR is antagonized the innate immune response may not be activated or may be activated at a reduced level compared to the absence of TLR antagonism.

### IV. METHODS OF MODULATING TLR PROTEINS

Provided herein are methods of modulating a TLR protein. In one aspect is a method of modulating a TLR protein by contacting the TLR protein with a compound described herein. The compound may be a compound of formula (I) or a compound as set forth in Table 1. The TLR protein is as described herein and may be a TLR4 protein. The activity of the TLR protein may be increased (i.e., agonized) upon contact with a compound described herein. The agonized TLR protein may activate the innate immune response. Activation of the TLR protein may activate NFκB protein. The modulation of the TLR protein may induce expression of IL-6 or IL-8. The activity of a TLR protein may be decreased (i.e., antagonized) upon contact with a compound described herein. The antagonized TLR protein may not activate the innate immune response, or the innate immune response may be decreased compared to the absence of TLR antagonism. The modulation may occur in a biological cell, that is to say the modulation may occur in vivo. The biological cell may form part of an organism, such as a human. The modulating may occur in vitro.

### V. METHODS OF TREATING DISEASE

Further provided herein are methods of treating or preventing a disease in a subject in need thereof. In one aspect is a method of treating or preventing a disease in a subject in need thereof by administering to the subject a therapeutically effective amount of a compound described herein. In another aspect is a method of treating a disease in a subject in need thereof by administering to the subject a therapeutically effective amount of a compound described herein. In yet another aspect is a method of preventing (e.g. prophylactic) a disease in a subject in need thereof by administering to the subject a prophylactic amount of a compound described herein.

The disease may be cancer. The cancer may be a cancer of the colorectal, breast, lung, liver, pancreas, lymph nodes, colon, prostate, brain, head and neck, skin, kidney or heart. The cancer may be a cancer of the colorectal, breast, lung, liver, pancreas, lymph nodes, colon, prostate, brain, head and neck, skin, kidney or heart. The cancer may be a hematopoietic neoplastic disorder involving hyperplastic/neoplastic cells of hematopoietic origin (e.g., arising from myeloid, lymphoid or erythroid lineages, or precursors cells thereof). The cancer may be from poorly differentiated acute leukemias, e.g., erythroblastic leukemia and acute megakaryoblastic leukemia. The cancer may be a myeloid disorder, e.g. acute promyeloid leukemia (APML), acute myelogenous leukemia (AML) and chronic myelogenous leukemia (CML). The cancer may be a lymphoid malignancy, e.g., acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). The cancer may be a malignant lymphoma, e.g., non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. The cancer may be a cell proliferative disorder, e.g., non-endocrine tumor or endocrine tumor. Representative non-endocrine tumors include adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

The disease may be an autoimmune disease. The autoimmune disease may be autoimmune encephalomyelitis, colitis, autoimmune insulin dependent diabetes mellitus (IDDM), Wegener granulomatosis, Takayasu arteritis, autoimmune hemolytic anemia, autoimmune hepatitis, autoimmune inner ear disease, Crohn's disease, inflammatory bowel disease, lupus, or multiple sclerosis.

The disease may be an infectious disease. The infectious disease may be a viral disease. The infectious disease may be acquired immunodeficiency syndrome, adenoviridae infections, alphavirus infections, arbovirus infections, Borna disease, bunyaviridae infections, caliciviridae infections, chickenpox, coronaviridae infections, coxsackievirus infections, cytomegalovirus infections, dengue, DNA Virus infections, ecthyma, contagious, encephalitis, arbovirus, Epstein-Barr virus infections, erythema infectiosum, hantavirus infections, hemorrhagic fevers, viral, hepatitis, viral, human, herpes simplex, herpes zoster, herpes zoster oticus, herpesviridae infections, infectious mononucleosis, influenza, e.g., in birds, swine, or humans, Lassa fever, measles, Molluscum contagiosum, mumps, paramyxoviridae infections, phlebotomus fever, polyomavirus infections, rabies, respiratory syncytial virus Infections, Rift Valley fever, RNA Virus Infections, rubella, slow virus diseases, smallpox, subacute sclerosing panencephalitis, tumor virus infections, warts, West Nile fever, virus diseases and Yellow Fever.

The infectious disease may be a bacterial infection. The bacterial infection may be a staphylococcal infection, e.g., methicillin-resistance Staphylococcus aureus (MRSA). The infectious disease may be anthrax, pertussis, Lyme disease, brucellosis, acute enteritis, community-acquired respiratory infection, nongonococcal urethritis, psittacosis, botulism, pseudomembranous colitis, gas gangrene, tetanus, diphtheria, urinary tract infection associated with E. coli, Traveler's diarrhea, hemorrhagic colitis, tularemia, bacterial meningitis, peptic ulcer, Legionnaire's disease, Pontiac fever, leptospirosis, listeriosis, leprosy, tuberculosis, mycoplasma pneumonia, gonorrhea, meningococcal disease, Waterhouse-Friderichsen syndrome, Rocky Mountain spotted fever, salmonellosis, bacillary dysentery, cystitis, otitis media, sinusitis, streptococcal pharyngitis, scarlet fever, rheumatic fever, impetigo, puerperal fever, necrotizing fasciitis, syphilis and cholera.

The disease may be an inflammatory disease or inflammation associated with a disease or injury. The inflammation may be associated with acne vulgaris, asthma, autoimmune diseases and disorders, celiac disease, chronic inflammation, chronic prostatitis, glomerulonephritis, hypersensitivities, inflammatory bowel disease, myopathy (e.g., in combination with systemic sclerosis, dermatomyositis, polymyositis, and/or inclusion body myositis), pelvic inflammatory disease, reperfusion injury, rheumatoid arthritis, sarcoidosis, transplant rejection, vasculitis, interstitial cystitis, atherosclerosis, allergies, leukocyte defects (e.g., Chediak-Higashi syndrome), chronic granulomatous disease, or cancer.

The subject may be a cancer patient having inflammation associated with cancer. As known in the art, inflammation plays a critical role in the microenvironment of tumors and contributes to proliferation, survival and migration. Balancing these activities, the immune system can suppress cancer. Accordingly, if an activated immune response suppresses cancer, then the inflammation arising from the cancer may be decreased. Thus, the subject may be a cancer patient, and administration of an effective amount of a compound described herein may result in an increase in the innate immune response, an increase in the activity of the adaptive immune system, and reduction in tumor size and associated inflammation.

The disease may be a pathogen invasion associated with inflammation. Thus, in such embodiments, when a compound described herein is administered, the innate immune response may be activated, and the pathogen may be neutralized, thereby reducing the associated inflammation. The pathogen may be a bacterial pathogen, and the infectious disease may be a disease described herein. The pathogen may be a viral pathogen, and the infectious disease may be a disease described herein.

The disease may be cancer. When the disease is cancer, the administered compound described herein may modulate the immune response in the subject having cancer by increasing an immune response to a cancer cell relative to the absence of said compound. Thus, in such embodiments, the elicited immune response kills the cancer cell. When the disease is an infectious disease, the administered compound described herein may modulate the immune response in the subject having an infectious disease by increasing an immune response to a pathogen relative to the absence of said compound. Thus, in such embodiments, the elicited immune response inactivates or kills the pathogen. When the disease is an autoimmune disease, the administered compound described herein may modulate the immune response in the subject having an autoimmune disease by decreasing an immune response to an endogenous antigen relative to the absence of said compound. The endogenous antigen may be associated with the autoimmune disease. The endogenous antigen may be associated with onset of the autoimmune disease or its symptoms. The endogenous antigen may be associated with progression of the autoimmune disease or its symptoms. When the disease is an inflammatory disease, or if the subject exhibits inflammation, the administered compound described herein may decreasing an immune response to the inflammation or the site of inflammation in said subject. The compound may have formula (I). The compound may be a compound set forth in Table 1. The modulation may result from modulation of a TLR. The modulation may result from a modulated immune response as described herein.

### VI. METHODS OF MODULATING IMMUNE RESPONSE

Provided herein are methods of modulating an immune response in a subject in need thereof. In one aspect, the method includes administering to the subject an effective amount of a compound described herein. The immune response may be increased in response to agonism of a TLR (e.g., TLR2, TLR3, TLR4, TLR5, TLR7, TLR8 or TLR9, preferably TLR4). The immune response may be decreased in response to antagonism of a TLR (e.g., TLR2, TLR3, TLR4, TLR5, TLR7, TLR8 or TLR9, preferably TLR4).

The subject may be a cancer patient and the modulation may include increasing an immune response to a cancer cell, relative to the absence of the compound. Thus, in such embodiments, a method for treating cancer in a patient in need thereof is provided as described herein. The method includes administering to the patient an effective amount of a compound described herein to the subject. The cancer is a cancer described herein.

The subject may be an infectious disease patient and the modulating may include increasing an immune response to a pathogen, relative to the absence of the compound. Thus, in such embodiments, a method for treating an infectious disease in a patient in need thereof is provided as described herein. The method includes administering to the patient an effective amount of a compound described herein. The infectious disease is an infectious disease described herein.

The subject may be an autoimmune disease patient and the modulating may include decreasing an immune response to an endogenous antigen causing the autoimmune disease, relative to the absence of the compound. Thus, in such embodiments, a method for treating an autoimmune disease in a patient in need thereof is provided as described herein. The method includes administering to the patient an effective amount of a compound described herein. The autoimmune disease is an autoimmune disease described herein.

Further provided herein are methods of decreasing inflammation (e.g. inflammatory disease or inflammation associated with a disease or injury) in a subject in need thereof. The method includes administering to the subject an effective amount of a compound described herein.

### VII. EXAMPLES

### Example 1

### Discovery of Substituted 4-Amino Quinazolines as Selective Toll-Like Receptr 4 Ligand

The first line of defense against microbial pathogens such as viruses, bacteria, fungi and protozoa is governed by the innate immune response, which includes barrier tissues and leukocytes. Many innate signaling pathways converge on the NF-κB family of transcription factors, which are critical in the regulation of these responses. A small molecular weight molecule that is an effective NF-κB activator could be incorporated into vaccine adjuvants as an innate immune activator. Adjuvants are added to antigens in vaccines to augment adaptive immune responses to poorly immunogenic antigens, and to increase protective antibody titers in at risk populations due to age (infants and the elderly), or disease (diabetes, liver failure). Adjuvants also facilitate the use of smaller doses of antigen, and enable effective immunization with fewer booster immunizations. Development of small molecular weight non-lipid ligands that are reactive in human cells has several advantages to address different immunological requirements for vaccines or immune therapeutics.

As part of studies on small molecules that can activate NF-κB signaling, we conducted a high through-put screening (HTS) campaign in which a commercially available library of over 170,000 compounds was screened in a human monocyte cell-based NF-κB activation assay. By utilizing a cell-based assay we theorized that we could identify small molecules that activated NF-κB through a broad range of mechanisms. Following the cluster enrichment analysis, 225 compounds were selected for further in vitro biological evaluation involving cytokine induction assays in primary cells, including human peripheral blood mononuclear cells (hPBMC), mouse splenocytes, mouse bone marrow derived dendritic cells (mBMDC) and mouse bone marrow derived macrophages (mBMDM). These cells were incubated in triplicate with each of the 225 compounds at a single concentration (1 μM for splenocytes and BMDC, 5 μM for all other mouse cells, and 5 μM for human cells) and the supernatants were tested for the presence of NF-κB dependent cytokines, IL-8 or IL-6, released from the human or mouse cells, respectively. Thirty-nine of the 225 compounds stimulated the human and mouse cells to secrete IL-8 or IL-6 above the detectable limit. To further confirm activity, these compounds were retested by stimulating hPBMC and mBMDC with titrated doses and assaying for IL-8 and IL-6, respectively.

Substituted 4-aminoquinazolines emerged as a potent class of compounds in activating human cells. As the Toll-like receptors (TLRs) are the most well understood and studied family of innate immune receptors these receptors were considered as likely targets for the HTS hit compounds. A few examples of pathogen-associated molecular patterns that are natural TLR ligands and their corresponding TLRs include: lipopeptides (TLR2), double-stranded RNA (TLR3), lipopolysaccharide (LPS, TLR4), bacterial flagellin (TLR5), guanine and uridine-rich singlestranded RNA (TLR7, 8), and hypo-methylated CpG rich DNA (TLR9). The specific innate receptor for the leading hit compound of the 4-aminoquinazolines was determined using primary cells from genetically modified mice, and mouse and human TLR reporter cells. Structure-activity relationship (SAR) studies of the substituted 4-aminoquinazolines class of ligands and their characterization as TLR4/MD-2 agonists are described herein. Within this scaffold cluster, a hit from the initial primary and secondary screens was a nitrophenyl-containing 4-aminoquinazoline bearing a phenoxy group on the nitrophenyl moiety (e.g. Compound 1a)

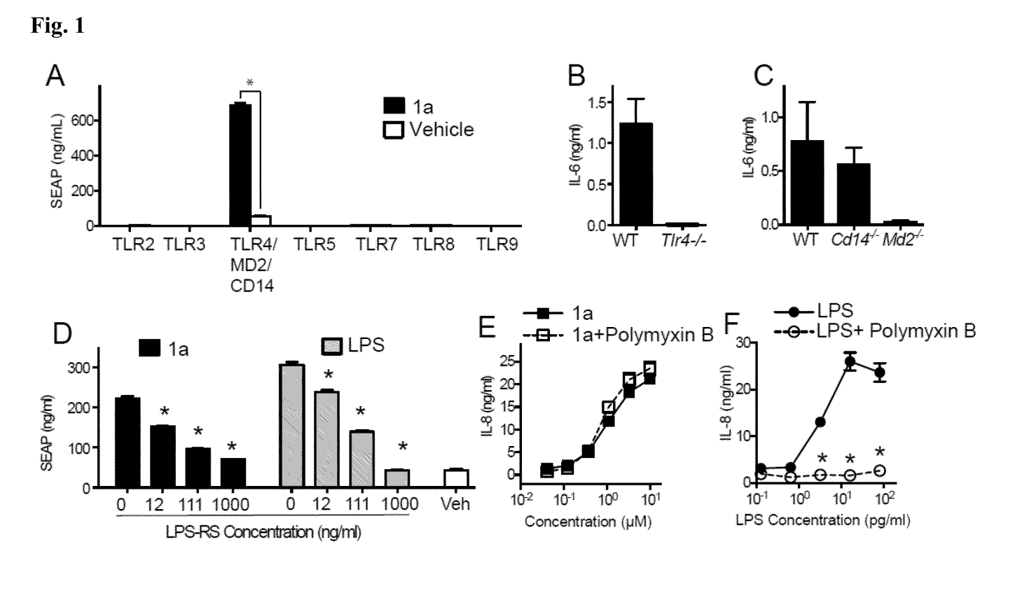

To identify the target receptor, we used HEK293 cells stably transfected with the following human TLRs (TLR2, TLR3, TLR4/MD-2/CD14, TLR5, TLR7, TLR8 or TLR9) along with an NF-κB activation reporter producing Secreted Alkaline Phosphatase (SEAP, FIG. 1A). Among the tested TLR transfected cells, only those expressing TLR4/MD-2/CD14 responded to hit compound 1a, as shown in FIG. 1A. To further confirm that TLR4 was indeed the receptor, compound 1a was assayed for IL-6 production in mBMDCs from wild type and TLR4 deficient mice (FIG. 1B). Genetic disruption of TLR4 completely abrogated IL-6 secretion induced by compound 1a. TLR4 signals through two distinct pathways, leading respectively to NF-κB dependent cytokines and type I interferon (IFN) production. Several naturally occurring TLR4 ligands and MPLA have been reported to require CD14 to activate the IFN regulatory pathway. The hTLR4 reporter cell line (FIG. 1A) also overexpresses hMD-2 and hCD14, which are TLR4 accessory proteins. Compound 1a, however, appeared not to be dependent on CD14, but dependent on MD-2 for IL-6 production, as demonstrated using mBMDC genetically deficient for CD14 or MD-2 (FIG. 1C).

The binding of compound 1a to the TLR4/MD-2/CD14 complex was further confirmed using a competitive antagonist for the TLR4 binding complex, LPS-RS (LPS from Rhodobacter sphaeroides). LPS-RS inhibited NF-κB activation in hTLR4/MD-2/CD14 transfectoma cells induced by compound 1a in a dose dependent manner, indicating that compound 1a bound to the TLR4 complex (FIG. 1D). Since TLR4/MD-2/CD14 was the receptor complex for the active compounds in this series, it was important to rule out the possibility that activity might have been caused by LPS contamination. Therefore, compound 1a (and all active derivatives) were assayed for LPS (endotoxin) levels using a commercially available detection system and found to contain less than 2 EU/lmol of endotoxin. To further exclude contamination, compound 1a was re-synthesized according to Scheme 1 following. Both samples of compound 1a displayed indistinguishable physicochemical and biological properties, indicating that the positive biological activity was not due to LPS or another contaminant. Furthermore, the IL-8 inducing ability of compound 1a was not reduced when assayed in the presence of polymyxin B, an LPS binding agent (FIG. 1E) although the same concentration of polymyxin B significantly suppressed IL-8 release by LPS stimulation (FIG. 1F).

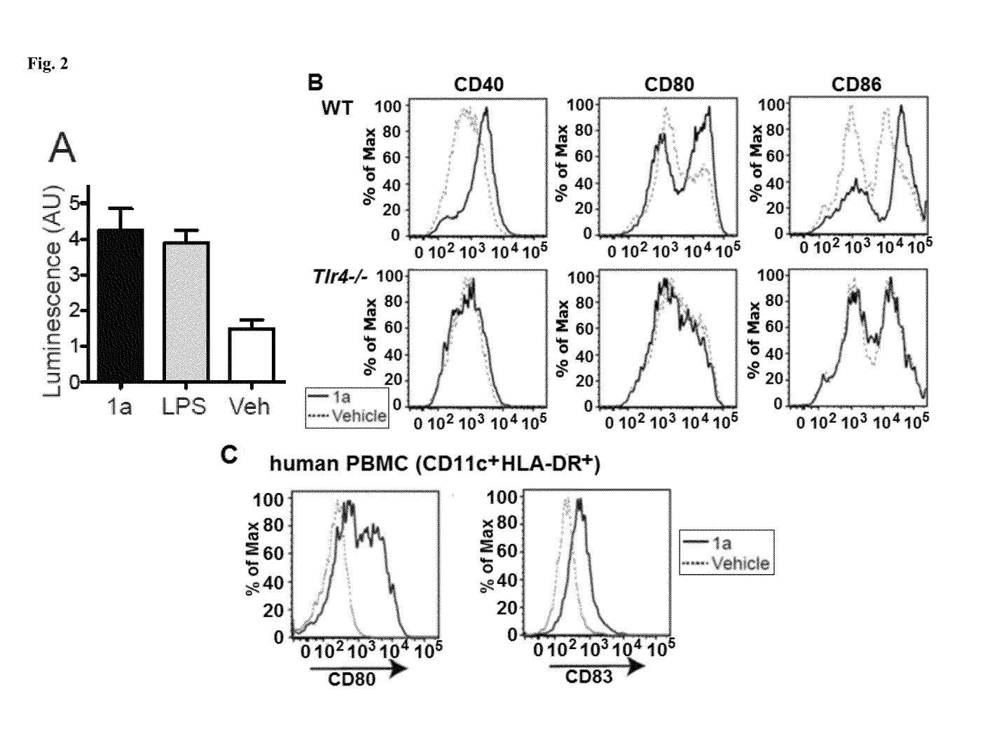

The engagement by active compounds, such as compound 1a, of the TLR4/MD-2/CD14 complex may result in signaling through the two pathways mentioned above. To evaluate whether the type I IFN pathway was activated, hPBMC were incubated with compound 1a or with LPS or vehicle overnight. The release of type I IFN in the culture supernatants was then determined in a type I IFN reporter cell assay. Compound 1a was found to induce the release of type I IFN comparable to that of LPS (FIG. 2A).

To evaluate whether treatment of DCs with this compound can induce maturation in these cells, mBMDC prepared from wild type or TLR4 deficient mice were exposed to compound 1a and then assayed for expression of co-stimulatory molecules CD40, CD80, and CD86 in the CD11c+ population (FIG. 2B). The expression of all three co-stimulatory molecules was increased in the wild type cells treated with compound 1a, but not in the TLR4 deficient cells. Induction of co-stimulatory molecules by compound 1a was also confirmed in human PBMC. After incubation of PBMC with compound 1a overnight, increased expression of CD80 and CD83 was observed in the DC-enriched (CD11c+HLA-DR+) gated population (FIG. 2C).

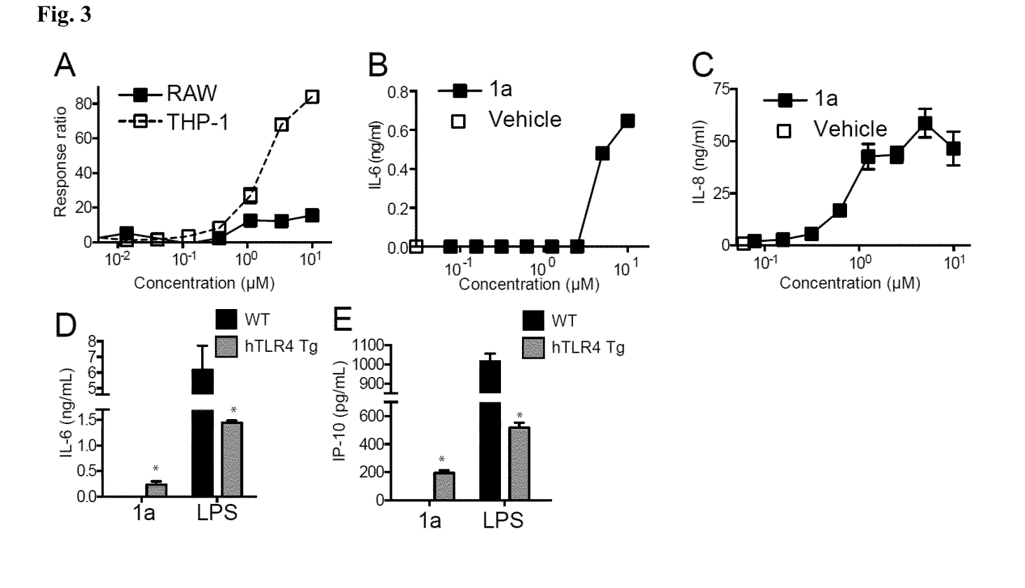

The original HTS of the compound library was conducted using THP-1 cells, a human monocytic leukemia cell line. For further in vivo immunological studies, mouse models are commonly used. It was therefore of interest to determine the relative level of immunoactivity in both mouse and human cells for the 4-aminoquinazolines. To examine this, RAW264.7 cells (a mouse macrophage cell line) or THP-1 cells, both containing a Fluorescence Resonance Energy Transfer (FRET) β-lactamase reporter gene construct downstream of an NF-κB promoter sequence, were incubated with graded concentrations of compound 1a for 18 h. The levels of NF-κB activation were determined, normalized to the level induced by LPS and expressed as percent activation (FIG. 3A). Compound 1a is significantly more potent in human THP-1 versus mouse RAW264.7 cells. Cytokine induction was evaluated by exposing mBMDC or hPBMC to graded concentrations of compound 1a and levels of IL-6 or IL-8, respectively, were determined by ELISA (FIGS. 3B and 3C). Results show that even at concentrations below 1 μM, compound 1a was able to induce detectable levels of IL-8 by hPBMC whereas higher concentrations were required to induce detectable levels of IL-6 by mBMDC, thus supporting the notion that compounds herein are more potent in human cells than in mouse cells. The viability of mouse BMDC and human PBMC following 18 h incubation with compound 1a (10 μM) was 117±2.6% and 112±2.1% by MTT assay, respectively, indicating that the lower activity of compound 1a was not due to its cytotoxicity. In fact, in vitro studies using BMDM prepared from transgenic mice that were genetically altered to express human TLR4/MD-2 and deficient for murine TLR4/MD-2 showed that compounds 1a and 1g in this series do indeed activate the transgenic human TLR4/MD-2 complex to a greater extent relative to mouse TLR4/MD-2 (FIGS. 3C, 3D, and 3E).

Without being bound by any particular theory, the 4-aminoquinazoline compound discovered herein also appears to bind to the TLR4/MD-2 complex. Accordingly, we examined the predicted binding mode(s) of compound 1a to the crystal structure of the human TLR4/MD-2 complex (PDB: 3fxi) using the docking program HEX,16 and AMPAC17 for optimization of the compound conformation. We selected the best configurations of 1a bound to this complex based on molecular surface shape complementarity and the most favorable intermolecular energy of interactions. Interestingly, the best docking position for 1a was within the LPS pocket.

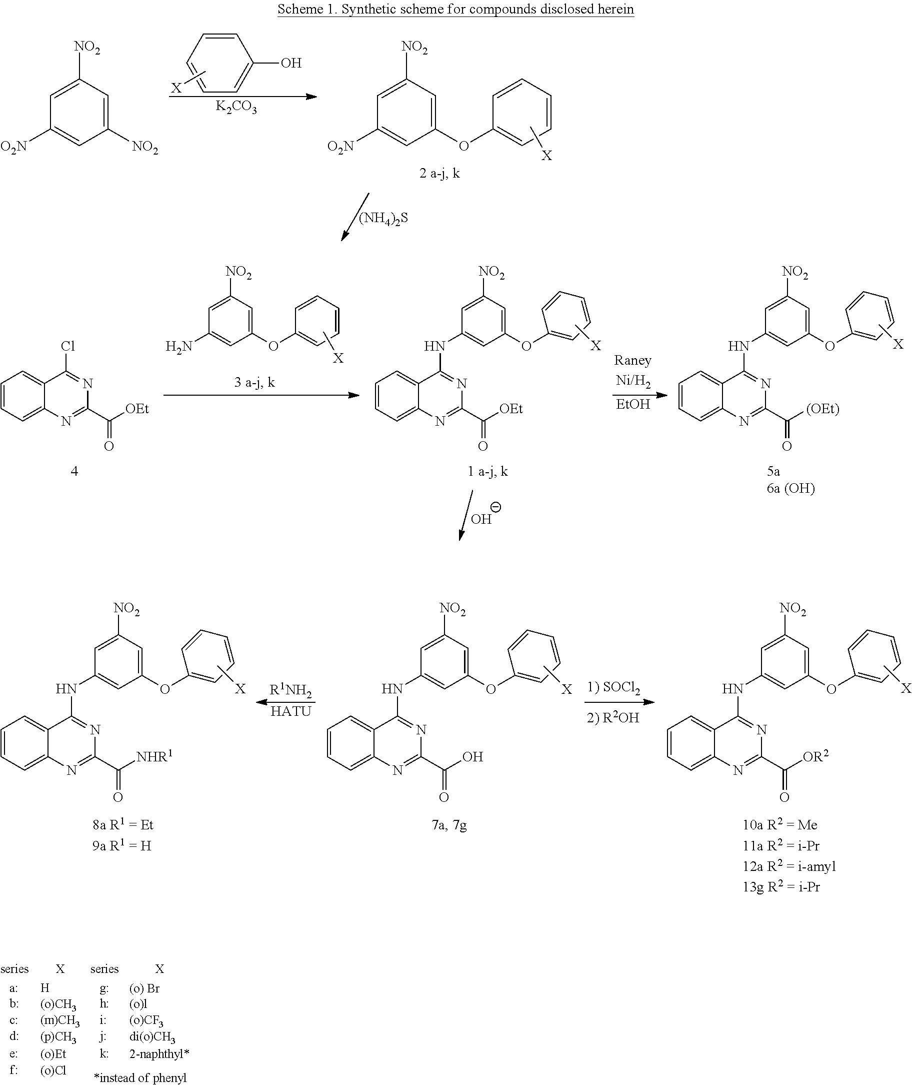

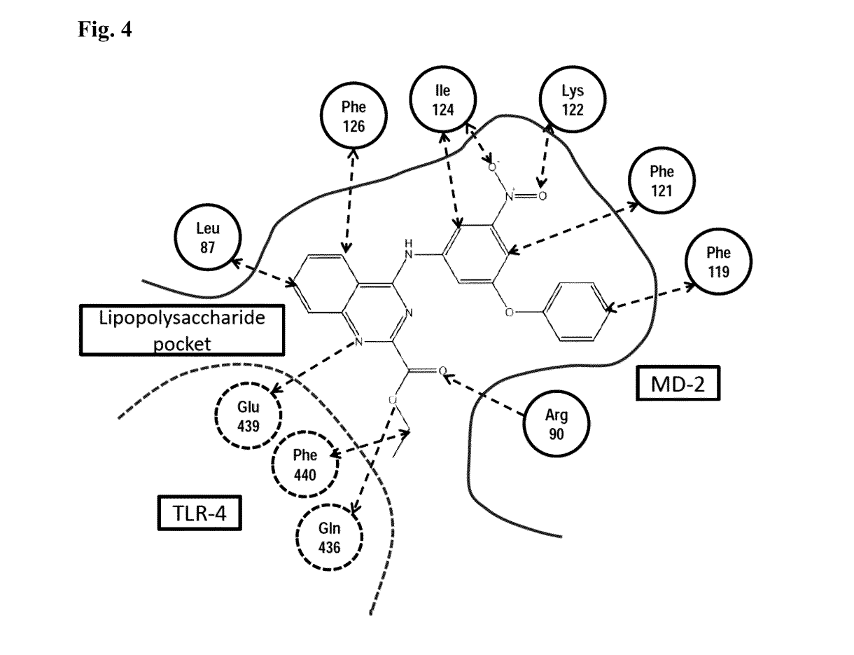

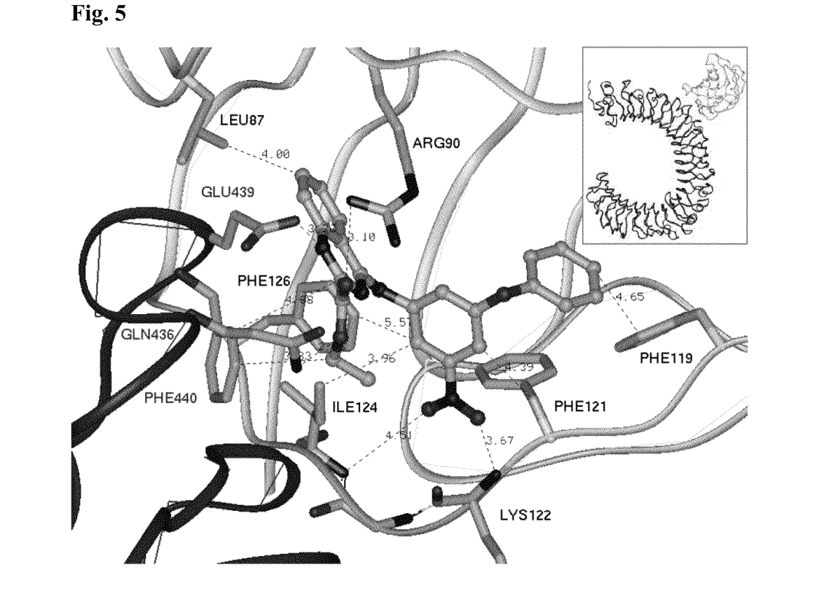

FIG. 4 shows the predicted binding mode of compound 1a in the TLR4/MD-2 model, while the set of binding interactions that may keep the compound in the MD-2 pocket bound to both TLR4 and MD-2 is depicted in FIG. 5. There is a set of possible hydrogen bonds formed by the residues Gln436 and Glu439 of TLR4 and Arg90 of MD-2. Noteworthy is the interaction of the two polar nitro oxygens of the compound with the backbone nitrogens of Ile124 and Lys122 of the MD-2 protein. The positive charge of the Lys122 provides a significant attractive force for a nitro oxygen in the human TLR4/MD-2 complex, whereas in the mouse complex, this residue is replaced by glutamic acid. Thus, the negative charge of the Glu122 in the mouse MD-2 causes an increase in repulsion energy as it encounters the negative oxygens of the nitro function in the compound and is likely at least partially responsible for the decreased activity of the compound in mouse cells relative to human cells. Also, there are several hydrophobic interactions with MD-2, involving residues Leu87, Phe126, Ile124, Phe121, and Phe119. Taken together, such interactions of the compound with two proteins can improve the free energy of complex formation by approximately 10 kcal/mol.

The above data indicate that compound 1a activates NF-κB preferentially through the human TLR4/MD-2 complex rather than through the murine TLR4/MD-2 complex, in a CD14 independent manner. Considering this selective activity, it was thought that compound 1a might serve as a useful chemical probe to investigate TLR4/MD-2 receptor-ligand specificity. Hence, compound 1a provided for additional structure-activity studies.

The primary HTS library contained a very small family of compounds in the 4-aminoquinazoline class and therefore provided only a limited indication of structural features important for activation of NF-κB. Substitutions at three sites were first undertaken to probe structural requirements and limitations with respect to optimization of cytokine induction in hPBMC and induction of NF-jB activation in TLR4 reporter cells (Table 1). While keeping all other structural features of hit compound 1a constant, we prepared a series of derivatives with variations at (1) the phenoxy moiety; (2) the nitro group; and (3) the ester function. The synthesis began with construction of the appropriately substituted nitroaniline as shown in Scheme 1. Trinitrobenzene was carefully reacted with appropriately substituted phenols according to the general method of Shevelev et al. to provide the corresponding dinitrophenoxy compounds 2a-j, and k. The dinitro intermediates were then partially reduced to the corresponding nitroanilines (3a-j, and k) using ammonium sulfide. These nitroanilines were condensed with the chloroquinazoline compound 4 to provide the corresponding substituted 4-aminoquinazolines 1a-j and k. Thus, we re-synthesized the original hit compound 1a by this method along with a variety of phenoxy derivatives substituted with alkyl and halogen groups. As indicated in Table 1, the results from assays using the TLR4 reporter cells and hPBMC suggest that when the phenoxy group in 1a is substituted in the ortho position by groups such as chloro (10, bromo (1g), iodo (1h), or methyl (ab), the activity is enhanced relative to hydrogen (values in the Table are normalized to LPS positive control=100). However, if the alkyl group is larger than methyl, such as ethyl (1e), a substantial loss of activity is observed. Likewise, if the aryloxy group is larger than phenoxy, such as naphthyloxy (1k), a loss of activity is also encountered. The importance of the nitro group in compound 1a for cytokine induction was investigated by reduction of the nitro to the primary amine compound (5a) using Raney nickel which resulted in complete loss of activity. Next the ester function was modified to include conversion to the free carboxylic acid (6a, 7a, 7g) and then to the carboxamide (9a) and the N-ethylcarboxamide (8a). Other esters were formed including the methyl (10a), isopropyl (11a) and isoamyl (12a) esters. The compound that combines the features of the ortho-bromophenoxy moiety and the isopropyl ester (13g) was also prepared.

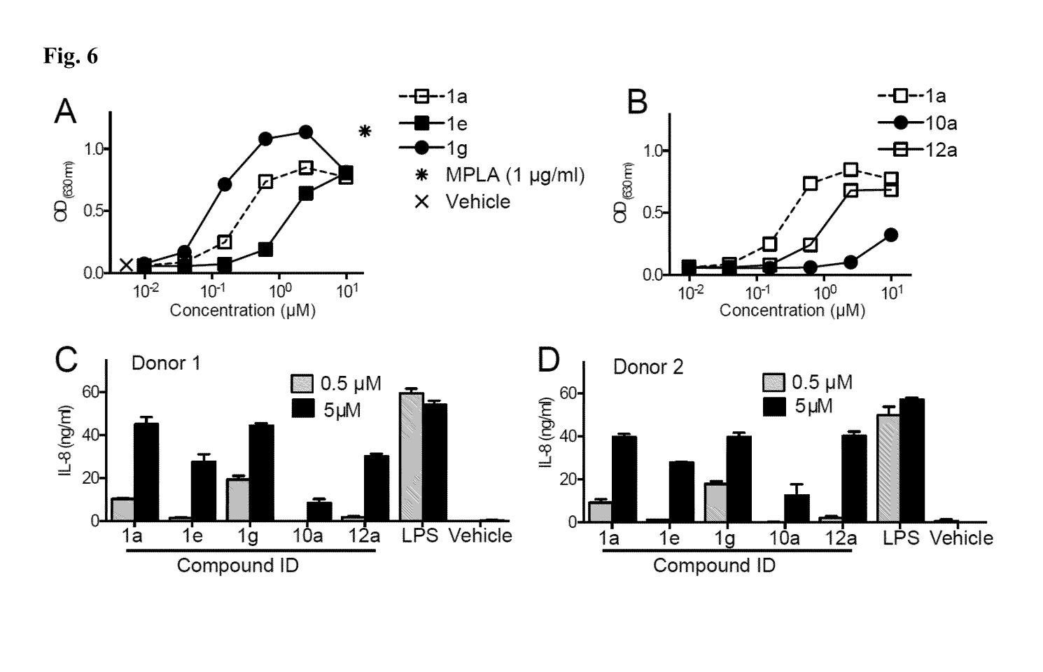

Besides a nitro group in the aminophenyl moiety for TLR4 binding activity, a comparison in hTLR4 reporter cells of selected compounds from the SAR studies for modifications at the ortho-position of the phenoxy moiety suggests that lower alkyl or halogen groups at this position are favored for activity (FIG. 6A). Likewise, for the ester function at the 2-position of the quinazoline ring, the ethyl ester is preferred over a smaller or larger alkyl group (FIG. 6B). In primary hPBMC, the same trends are apparent, particularly at the lower, more physiologically relevant concentration of 0.5 1M (FIGS. 6C and 6D).

In the course of an HTS designed to identify activators of innate immunity, a series of substituted 4-aminoquinazolines was discovered as selective TLR4 ligands. Small molecules of this class are unique among TLR4 activators in that they are ‘non-lipid-like’. Structure-activity evaluation in both mouse and human cells revealed that this series of small molecules activates the human TLR4/MD-2 complex more potently than the corresponding mouse complex and this activation was observed to be MD-2-dependent and CD14-independent. Interestingly, a recent report describes a natural product, Euodenine A, that activates human TLR4 more potently than the corresponding mouse receptor and shares a similar immunologic profile with the 4-aminoquinazolines described here. The 4-aminoquinazolines species preference phenomenon could be explained, at least in part, and without being bound by any particular theory, by the observation that certain amino acid residues of the MD-2 proteins of the TLR4/MD-2 complex vary in mouse versus human at critical interaction binding sites for the 4-aminoquinazoline compounds. Indeed, a simple change in residue 122 of MD-2 from Lys in human to Glu in mouse is predicted to reverse the electrostatic influence of binding of the nitro oxygen(s) of, for example, the active compound 1a. The result is an attractive force in the human complex for the compound, but a repulsive force at the same site in the mouse complex.

Initial SAR studies revealed several findings. First, the nitro group on the phenylamino moiety is useful for activity since reduction to amino abrogated the activity. Indeed, computational studies showed that interactions at the predicted binding site of MD-2 for the nitro compound were much more favorable (by as much as 6.1 kcal/mol total energy improvement in interaction energy) compared to the corresponding amino compound. Aromatic or heteroaromatic nitro groups are well known in drug design. Examples are the 5-nitrofurans and 2- and 5-nitroimidazoles. These drugs are used as therapeutic agents against a variety of protozoan and anaerobic bacterial infections in humans and animals. However, the compounds reported here represent the first known class of nitro-containing small molecules that can activate a human innate immune receptor. Second, a small alkyl or halogen group at the ortho position of the phenoxy moiety enhances activity. Third, a lower alkyl ester at the 2-position of the quinazoline ring appears to be favored for activity, while some compounds having the corresponding carboxylic acid form or unsubstituted amide are not active. Interestingly none of the substitutions increased the activity of the compound to preferentially stimulate murine rather than human cells, a finding opposite from that observed with certain other small molecule Lead optimization studies to improve the activity of the compounds using computational methods are currently underway in our laboratories. Optimized lead compounds in the 4-aminoquinazoline class that stimulate innate immune cells with minimal toxicity may be useful as adjuvants or immunotherapeutic agents.

Materials. Reagents were purchased as at least reagent grade from Sigma-Aldrich (St. Louis, Mo.) unless otherwise specified and used without further purification. Solvents were purchased from Fischer Scientific (Pittsburgh, Pa.) and were either used as purchased, or redistilled with an appropriate drying agent. The HTS compound library was obtained from University of California San Francisco Small Molecule Discovery Center (San Francisco, Calif.). Commercial HTS services were provided by Invitrogen (Madison, Wis.). Hit compoundla wasrepurchased from Life Chemicals (Burlington, ON, Canada). Endotoxin levels of active compounds were measured withEndosafe® —PTS (Charles River, Wilmington, Mass.) and found to have less than 2 EU/μmol.

The 3D structures were prepared and optimized using the AMPAC semi-empirical quantum chemistry program (Accelrys, San Diego, Calif.). Compounds used for structure-activity studies were synthesized according to methods described in Results section 3.2.

Instrumentation. Analytical TLC was performed using precoated TLC silica gel 60 F₂₅₄ aluminum sheets purchased from EMD (Gibbstown, N.J.) and visualized using UV light. Flash chromatography was carried out using EMD silica gel 60 (40-63 μm) or with a Biotagelsolera One (Charlotte, N.C.) system using the specified solvent. Reactions were monitored using an Agilent 1100 LC/MSD (Santa Clara, Calif.) with either a Supelco Discovery HS C18 column (Sigma-Aldrich) or an Onyx Monolithic C18 (Phenomenex, Torrance, Calif.) with purity above 97% by percent area for all final compounds except as noted for compounds 1i, 1k and 12a. All synthesized compounds and intermediates were analyzed by high resolution MS using an Agilent 6230 ESI-TOFMS (Santa Clara, Calif.). 1H NMR spectra were obtained on a Varian Mercury 300 (Varian, Inc., Palo Alto, Calif.). The chemical shifts are expressed in parts per million (ppm) using suitable deuterated NMR solvents in reference to TMS at 0 ppm.

High throughput screen study design. A library of compounds was acquired consisting of about 170,000 chemical entities from eight suppliers (https://smdc.ucsf.edu). The library was screened in three phases using the fluorescence resonance energy transfer (FRET)-based THP-1 GeneBlazer (Life Technologies) human monocytic leukemia cell line, which contained a β-lactamase reporter gene under the control of the NF-κB response element that had been stably integrated into the cells (see e.g, website tools.lifetechnologies.com/content/sfs/manuals/CellSensor_NF-kBbla-THP1_man.pdf). All screens were performed in activator mode using lipopolysaccharide (LPS) as a positive control, achieving typical Z′ values above 0.75.¹ The three-phase screening process consisted of 1) a pilot screen of about 10,000 compounds selected as representative of the entire primary library; 2) the primary screen of the entire library; and 3) a confirmation screen of about 2,000 hits found in the primary screen. Compounds identified as active in two screens were considered to be confirmed hits. An analysis of the cluster enrichment methods for hit selection has been recently reported.²

Measurement of NF-κB activation using CellSensor® NF-κB-bla Raw 264.7 and THP-1 cell lines. CellSensor® NFκB-bla RAW 264.7 and THP-1 cell lines were purchased from Life Technologies. CellSensor® NF-κB-bla RAW 264.7 were harvested, resuspended in Assay Medium (99.5% OptiMEM; 0.5% dialyzed FBS; 0.1 mM non-essential amino acids; 1 mM sodium pyruvate; 10 mM HEPES pH 7.3; 100 U/ml penicillin; 100 μg/ml streptomycin) and plated in 96-well plates at 90 μl (5×10⁴ cells) per well. 10 μl of the 10× solution of compounds in the assay medium was added to the wells. Cells were then incubated for approximately 16 h in 5% CO₂ at 37° C., after which 20 μl of 6xLiveBLAzer™ FRET B/G Substrate (CCF4-AM) Mixture (prepared according to the manufacturer's instructions) was added to each well. Plates were incubated at room temperature in the dark for 3 to 5 h. Fluorescence was measured on a Tecan Infinite M200 plate reader (Männedorf, Switzerland) at an excitation wavelength of 405 nm, and emission wavelengths of 465 nm and 535 nm.

The assay with CellSensor® NF-κB-bla THP-1 cells was performed almost identically to that with CellSensor® NF-κB-bla RAW 264.7 cells, except that cells were harvested, resuspended, and plated in RPMI (plus 10% dialyzed FBS, 0.1 mM non-essential amino acids, 1 mM sodium pyruvate, 100 U/ml penicillin and 100 μg/ml streptomycin) and incubated with drugs for only 5 h prior to addition of the 6× substrate mixture. Plates were then incubated for 2 h prior to measurement of fluorescence.

Animals. Seven to 9 week-old C57BL/6 (wild type, WT) and Cd14^{-/-} (C57BL/6 background) mice were purchased from the Jackson Laboratories (Bar Harbor, Mass.). Tlr4^{-/-} mice (C57BL/6 background) were a gift from Dr. Shizuo Akira (Osaka University, Japan) and were backcrossed for 10 generations onto the C57BL/6 background at University of California, San Diego (UCSD). MD-2^{-/-} mice (C57BL/6 background) were originally generated by Dr. K. Miyake (University of Tokyo, Tokyo, Japan) and were kindly provided by Dr. L. Hajjar (University of Washington, Seattle, Wash.). Human TLR4 transgenic mice on murine TLR4/MD-2 null C57BL/6 background were kindly provided by Dr. L. Hajjar.³ All animal experiments were approved by the UCSD Institutional Animal Care and Use Committee.

In vitro cytokine induction in bone marrow derived dendritic cells (BMDC) and macrophages (BMDM). BMDC or BMDM were prepared from C57BL/6 mice as described.⁴ BMDC or BMDM (10⁵ cells per well) were plated in 96-well plates in 200 μl complete RPMI1640 (Omega Scientific, Tarzana, Calif.) supplemented with 10% fetal calf serum (Sigma Aldrich), 100 U/mL penicillin and 100 μg/mL streptomycin (Life Technologies). The cells were incubated with graded concentrations of the compounds for 18 h at 37° C., 5% CO₂. After 18 h incubation, the cell culture supernatants were collected. LPS (100 ng/mL, L2654, E coli; B6, Sigma) or MPLA (1 μg/mL synthetic MPLA, Invivogen) were used as positive controls. The levels of IL-6 or IP-10 in the culture supernatants were determined by ELISA (BD Biosciences, La Jolla, Calif.).⁵ Finally, all levels of cytokine release were normalized to those of LPSstimulation within each experiment, set at 100.

In vitro assays using TLR reporter cell lines. Murine or humanTLR4 HEK Blue™ cells (Invivogen, 2.5×10⁴ cells per well of a 96 well plate), or NF-κB/SEAPorter™ HEK 293 cells (Imgenex, San Diego, Calif.) for human TLR2, TLR3, TLR5, TLR7, TLR8, or TLR9 (5×10⁴ cells per well of 96 well plate) were incubated with graded doses of the test compound. The culture supernatants were harvested after a 20-24 h incubation period. Secreted alkaline phosphatase (SEAP) activity in the supernatants was determined by a colorimetric assay, using either the SEAPorter Assay Kit (Imgenex, San Diego, Calif.), with absorbance read at 405 nm, or QuantiBlue (Invivogen), with absorbance read at 630 nm. In some experiments, graded concentrations of LPS-RS (Rhodococcus sphaeroides, Invivogen) were used to test TLR4-specific binding of the hit compound.

In vitro activities in human peripheral blood mononuclear cells (PBMC). Human PBMC were isolated from buffy coats obtained from the San Diego Blood Bank (San Diego, Calif.) as described previously.^{5,6} PBMC (1×10⁶/mL) were incubated with various compounds in complete RPMI (Omega Scientific) for 18 h at 37° C., 5% CO₂ and culture supernatants were collected. The levels of IL-8 in the supernatants were determined by ELISA (BD Biosciences).

Type I interferon (IFN) assay. L929 cells stably expressing an IFN sensitive response element (ISRE) luciferase reporter construct were kindly provided by Dr. B. Beutler (UT Southwestern, Dallas, Tex.)⁷. The bioactivity oftype I IFN in hPBMC supernatants was measured by luciferase assay using L929-ISRE cells as described previously.⁷ L929-ISRE cells were plated at 5×10⁴ cells per well inDulbecco's Modified Eagle Medium (Life Technologies) supplemented with 10% FCS, 100 U/mL penicillin and 100 μg/mL streptomycinin a 96-well white-walled clear-bottom plate and incubated overnight. Thus, 50 μL of supernatant was incubated with L929-ISRE cells in 50 μL of DMEM for 6 h. The luciferase activities were measured by GloBrightLuciferase Assay Buffer (Promega, Madison, Wis.).

TABLE 1
Activity of substituted 4-aminoquinazolines in hTLR4 transfectoma and
primary human PBMC.

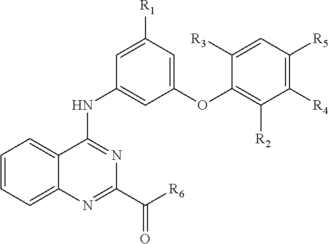

| | | | | | | | hTLR4^{a} | PBMC^{b} | HepG2 |
|---|---|---|---|---|---|---|---|---|---|
| Cmpd | R₁ | R₂(o) | R₃(o2) | R₄(m) | R₅(p) | R₆ | 0.5 μM | 0.5 μM | MT^{c} |
| LPS | | | | | | | 100 | 100 | 100.9 |
| 1a | NO2 | H | H | H | H | OC2H5 | 49.1 | 22.2 | 93.5 |
| 1b | NO2 | CH3 | H | H | H | OC2H5 | 45.5 | 20.5 | 96.7 |
| 1c | NO2 | H | H | CH3 | H | OC2H5 | 14.5 | 5.8 | 109.9 |
| 1d | NO2 | H | H | H | CH3 | OC2H5 | 0.5 | 0.4 | 108.8 |
| 1e | NO2 | Et | H | H | H | OC2H5 | 4.2 | 3.2 | 98.9 |
| 1f | NO2 | Cl | H | H | H | OC2H5 | 73.9 | 27.3 | 107.4 |
| 1g | NO2 | Br | H | H | H | OC2H5 | 79.0 | 35.6 | 90.8 |
| 1h | NO2 | I | H | H | H | OC2H5 | 65.5 | 34.2 | 93.8 |
| 1i | NO2 | CF3 | H | H | H | OC2H5 | 58.7 | 26.5 | 67.1 |
| 1j | NO2 | CH3 | CH3 | H | H | OC2H5 | 0.6 | 0.0 | 106.3 |
| 1k | NO2 | H | H | See d | See d | OC2H5 | 0.8 | 0.0 | 111.4 |
| 5a | NH2 | H | H | H | H | OC2H5 | 0.6 | 0.3 | 106.1 |
| 6a | NH2 | H | H | H | H | OH | 1.1 | 0.3 | 105.0 |
| 7a | NO2 | H | H | H | H | OH | 0.6 | 0.5 | 84.5 |
| 7g | NO2 | Br | H | H | H | OH | 1.6 | 0.0 | 96.3 |
| 8a | NO2 | H | H | H | H | NHC2H5 | 2.2 | 1.5 | 107.4 |
| 9a | NO2 | H | H | H | H | NH2 | 0.0 | 0.3 | 78.4 |
| 10a | NO2 | H | H | H | H | OCH3 | 0.4 | 0.2 | 116.4 |
| 11a | NO2 | H | H | H | H | O- | 7.4 | 2.1 | 65.6 |
| | | | | | | Isopropyl | | | |
| 12a | NO2 | H | H | H | H | O-Isoamyl | 8.4 | 2.2 | 63.4 |
| 13g | NO2 | Br | H | H | H | O- | 67.8 | 19.2 | 106.5 |
| | | | | | | Isopropyl | | | |

Footnotes:
^{a}Average Quantiblue OD₆₃₀ of hTLR4 HEK Blue cells incubated with LPS (100 ng/mL) was 1.22 ± 0.17.
^{b}Average IL-8 release by 100 ng/mL LPS was 25.9 ± 5.6 ng/mL.
^{c}MTT assay data (compounds tested at 10 μM) were normalized with OD₅₇₀₋₆₅₀ of vehicle treatment in the related experiments. The average MTT reading for HepG2 cells treated with vehicle was 1.326 ± 0.294.
^{d}2-Naphthol instead of phenol.

Chemical Procedures and Compounds: General Procedure A for the Synthesis of Compounds 2 a-j, k. 1,3,5-Trinitrobenzene (1 equiv) was added to a mixture of the substituted phenol (1 equiv), and fine-powdered K₂CO₃ (1 equiv) in N-methyl-2-pyrrolidone at 80° C. and monitored by TLC. The product was purified by automated silica gel column chromatography (normal phase, 2 to 20% ethyl acetate in hexanes gradient).

General Procedure B for the Synthesis of Compounds 3 a-j, k. Ammonium sulfide (20% solution) was added to a solution of the dinitro-derivative in ethanol with stirring at 75° C. The reaction was monitored by TLC until starting material disappeared (about 1 h). The product was purified by automated silica gel column chromatography (normal phase, 5 to 35% ethyl acetate in hexanes gradient).

General Procedure C for the Synthesis of Compounds 1 a-j, k. A mixture of compound 4 (1.1 equiv), and the nitroaniline derivative (l equiv) was suspended in 10% DMF in water and stirred at 90° C. for 2 h. The solid was filtered off, washed with cold water and purified by normal phase column chromatography (40 to 90% ethyl acetate in hexanes gradient).

Ethyl 4-((3-nitro-5-phenoxyphenyl)amino)quinazoline-2-carboxylate (1a). Compound 4 (50 mg, 0.21 mmol), and compound 3a (44 mg, 0.19 mmol) were reacted according to General Procedure C to yield 1a (71%). HRMS calcd for C₂₃H₁₁N₄O₅Na (M+Na)+, 453.1171; found, 453.1170. ¹H NMR (300 MHz, DMSO-d₆) δ ppm 1.36 (t, J=7.15 Hz, 3H) 4.37 (q, J=7.06 Hz, 2H) 7.23 (d, J=8.25 Hz, 1H) 7.28 (t, J=7.20 Hz, 3H) 7.51 (t, J=7.70 Hz, 1H) 7.47 (s, 3H) 7.79-7.89 (m, 1H) 8.02 (d, J=3.58 Hz, 2H) 8.38 (s, 1H) 8.67 (d, J=7.70 Hz, 1H) 9.06 (s, 1H) 10.38 (s, 1H).

Ethyl 4-((3-nitro-5-(o-tolyloxy)phenyl)amino)quinazoline-2-carboxylate (1b). Compound 4 (20 mg, 0.08 mmol), and compound 3b (19 mg, 0.08 mmol) were reacted according to General Procedure C to yield 1b (75%). HRMS calcd for C₂₄H₂₁N₄O₅ (M)+, 445.1506; found, 445.1505. ¹H NMR (300 MHz, DMSO-d₆) δ ppm 1.38 (t, J=7.20 Hz, 3H) 2.22 (s, 3H) 4.39 (q, J=7.06 Hz, 2H) 7.15 (d, J=7.70 Hz, 1H) 7.24 (t, J=7.40 Hz, 1H) 7.29-7.38 (m, 2H) 7.43 (d, J=7.40 Hz, 1H) 7.82 (m, J=8.30, 4.10, 4.10 Hz, 1H) 7.94-8.06 (m, 2H) 8.26 (t, J=2.20 Hz, 1H) 8.66 (d, J=8.25 Hz, 1H) 9.03 (t, J=2.20 Hz, 1H) 10.35 (s, 1H).

Ethyl 4-((3-nitro-5-(m-tolyloxy)phenyl)amino)quinazoline-2-carboxylate (1c). Compound 4 (24 mg, 0.10 mmol), and compound 3c (22.5 mg, 0.09 mmol) were reacted according to General Procedure C to yield 1c (70%). HRMS calcd for C₂₄H₂₁N₄O₅ (M)+, 445.1506; found, 445.1505. ¹H NMR (300 MHz, DMSO-d₆) δ ppm 1.36 (t, J=7.15 Hz, 3H) 2.35 (s, 3H) 4.37 (q, J=7.00 Hz, 2H) 6.90-7.15 (m, 3H) 7.37 (t, J=7.84 Hz, 1H) 7.44 (t, J=2.10 Hz, 1H) 7.82 (ddd, J=8.30, 4.90, 3.30 Hz, 1H) 7.93-8.07 (m, 2H) 8.35 (t, J=2.06 Hz, 1H) 8.66 (d, J=8.25 Hz, 1H) 9.06 (t, J=2.06 Hz, 1H) 10.35 (s, 1H).

Ethyl 4-((3-nitro-5-(p-tolyloxy)phenyl)amino)quinazoline-2-carboxylate (1d). Compound 4 (25 mg, 0.11 mmol), and compound 3d (23 mg, 0.1 mmol) were reacted according to General Procedure C to yield 1d (68%). HRMS calcd for C₂₄H₂₁N₄O₅ (M)+, 445.1506; found, 445.1505. ¹H NMR (300 MHz, DMSO-d₆) δ ppm 1.37 (t, J=7.15 Hz, 3H) 2.35 (s, 3H) 4.38 (q, J=7.24 Hz, 2H) 7.12 (d, J=8.53 Hz, 2H) 7.31 (d, J=8.53 Hz, 2H) 7.42 (t, J=2.20 Hz, 1H) 7.83 (ddd, J=8.46, 5.02, 3.30 Hz, 1H) 7.91-8.08 (m, 2H) 8.32 (t, J=2.20 Hz, 1H) 8.66 (d, J=8.25 Hz, 1H) 9.05 (t, J=2.06 Hz, 1H) 10.35 (s, 1H).

Ethyl 4-((3-(2-ethylphenoxy)-5-nitrophenyl)amino)quinazoline-2-carboxylate (1e). Compound 4 (20 mg, 0.09 mmol), and compound 3e (20 mg, 0.08 mmol) were reacted according to General Procedure C to yield 1e (71%). HRMS calcd for C₂₅H₂₃N₄O₅ (M)+, 459.1663; found, 459.1664. ¹H NMR (300 MHz, DMSO-d₆) δ ppm 1.17 (t, J=7.56 Hz, 3H) 1.37 (t, J=7.15 Hz, 3H) 2.61 (q, J=7.70 Hz, 2H) 4.39 (q, J=7.52Hz, 2H) 7.13 (d, J=8.53 Hz, 1H) 7.22-7.39 (m, 3H) 7.45 (d, J=8.80 Hz, 1H) 7.77-7.90 (m, 1H) 8.02 (d, J=3.58 Hz, 2H) 8.29 (s, 1H) 8.67 (d, J=7.98 Hz, 1H) 9.04 (s, 1H) 10.36 (s, 1H).

Ethyl 4-((3-(2-chlorophenoxy)-5-nitrophenyl)amino)quinazoline-2-carboxylate (1f). Compound 4 (18 mg, 0.08 mmol), and compound 3f (18 mg, 0.07 mmol) were reacted according to General Procedure C to yield 1f (70%). HRMS calcd for C₂₃H₁₈Cl N₄O₅ (M)+, 465.0960; found, 465.0961. ¹H NMR (300 MHz, DMSO-d₆) δ ppm 1.36 (t, J=7.01Hz, 3H) 4.38 (q, J=7.15 Hz, 2H) 7.24-7.44 (m, 3H) 7.49 (t, J=8.00 Hz, 1H) 7.71 (d, J=8.25 Hz, 1H) 7.83 (dt, J=8.11, 4.19 Hz, 1H) 7.90-8.09 (m, 2H) 8.32 (s, 1H) 8.66 (d, J=8.25 Hz, 1H) 9.08 (s, 1H) 10.37 (s, 1H).

Ethyl 4-((3-(2-bromophenoxy)-5-nitrophenyl)amino)quinazoline-2-carboxylate (1 g). Compound 4 (30 mg, 0.12 mmol), and compound 3g (35 mg, 0.11 mmol) were reacted according to General Procedure C to yield 1g (74%). HRMS calcd for C₂₃H₁₈BrN₄O₅ (M)+, 509.0455; found, 509.0454. ¹H NMR (300 MHz, DMSO-d₆) δ ppm 1.37 (t, J=7.15 Hz, 3H) 4.39 (q, J=6.97 Hz, 2H) 7.39 (s, 1H) 7.30 (t, J=7.40 Hz, 1H) 7.38 (d, J=6.88 Hz, 1H) 7.53 (t, J=7.40 Hz, 1H) 7.75-7.91 (m, 2H) 7.94-8.10 (m, 2H) 8.31 (s, 1H) 8.66 (d, J=8.25 Hz, 1H) 9.10 (s, 1H) 10.38 (s, 1H).

Ethyl 4-((3-(2-iodophenoxy)-5-nitrophenyl)amino)quinazoline-2-carboxylate (1h). Compound 4 (30 mg, 0.12 mmol), and compound 3h (35 mg, 0.11 mmol) were reacted according to General Procedure C to yield 1h (68%). HRMS calcd for C₂₃H₁₈IN₄O₅ (M)+, 557.0316; found, 557.0315. ¹H NMR (300 MHz, DMSO-d₆) δ ppm 1.38 (t, J=7.20 Hz, 3H) 4.39 (q, J=7.00 Hz, 2H) 7.13 (t, J=7.70 Hz, 1H) 7.30 (d, J=8.25 Hz, 1H) 7.36 (br. s., 1H) 7.53 (t, J=7.70 Hz, 1H) 7.84 (ddd, J=7.70, 4.00, 3.80 Hz, 1H) 7.94-8.11 (m, 3H) 8.30 (br. s., 1H) 8.67 (d, J=8.50 Hz, 1H) 9.11 (s, 1H) 10.38 (s, 1H).

Ethyl 4-((3-nitro-5-(2-(trifluoromethyl)phenoxy)phenyl)amino)quinazoline-2-carboxylate (1i). Compound 4 (15 mg, 0.06 mmol), and compound 3i (17 mg, 0.06 mmol) were reacted according to General Procedure C to yield 1i (65%) with purity >90%. HRMS calcd for C₂₄H₁₈F₃N₄O₅ (M)+, 499.1224; found, 499.1223. ¹H NMR (400 MHz, DMSO-d₆) δ ppm 1.33 (t, J=7.00 Hz, 1H) 4.36 (q, J=7.00 Hz, 2H) 7.36 (d, J=8.07 Hz, 1H) 7.47 (t, J=7.33 Hz, 1H) 7.52 (t, J=2.02Hz, 1H) 7.77 (t, J=7.88 Hz, 1H) 7.80-7.87 (m, 1H) 7.89 (d, J=7.70 Hz, 1H) 7.98-8.06 (m, 2H) 8.45 (t, J=1.83 Hz, 1H) 8.67 (d, J=8.43 Hz, 1H) 9.10 (t, J=1.65 Hz, 1H) 10.39 (s, 1H).

Ethyl 4-((3-(2,6-dimethylphenoxy)-5-nitrophenyl)amino)quinazoline-2-carboxylate (1j). Compound 4 (15 mg, 0.06 mmol), and compound 3j (15 mg, 0.06 mmol) were reacted according to General Procedure C to yield 1j (69%). HRMS calcd for C₂₅H₂₃N₄O₅ (M)+, 459.1663; found, 459.1662. ¹H NMR (300 MHz, DMSO-d₆) δ ppm 1.39 (t, J=7.40 Hz, 3H) 2.14 (s, 6H) 4.40 (quin, J=6.46 Hz, 2H) 7.11-7.30 (m, 4H) 7.75-7.92 (m, 1H) 8.01 (d, J=3.58 Hz, 2H) 8.09 (s, 1H) 8.65 (d, J=8.25 Hz, 1H) 9.01 (s, 1H) 10.33 (s, 1H).

Ethyl 4-((3-(naphthalen-2-yloxy)-5-nitrophenyl)amino)quinazoline-2-carboxylate (1k). Compound 4 (15 mg, 0.06 mmol), and compound 3k (17 mg, 0.06 mmol) were reacted according to General Procedure C to yield 1k (65%) with purity >90%. HRMS calcd for C₂₇H₂₀N₄O₅Na (M+Na)+, 503.1326; found, 503.1327. HRMS calcd for C₂₇H₂₀N₄O₅Na (M+Na)+, 503.1326; found, 503.1327. ¹H NMR (500 MHz, DMSO-d₆) δ ppm 1.33 (t, J=7.09 Hz, 3H) 4.36 (q, J=7.09 Hz, 2H) 7.42 (d, J=9.05 Hz, 1H) 7.46-7.58 (m, 2H) 7.66 (s, 1H) 7.75-7.83 (m, 2H) 7.84-7.92 (m, 2H) 7.93-8.02 (m, 2H) 8.05 (d, J=8.80 Hz, 1H) 8.39 (br. s., 1H) 8.62 (d, J=8.31Hz, 1H) 9.11 (br. s., 1H) 10.34 (br. s., 1H).

Ethyl 4-((3-amino-5-phenoxyphenyl)amino)quinazoline-2-carboxylate (5a). Compound 1a (65 mg, 0.15 mmol), was shaken with Raney nickel in ethanol. Hydrogen gas was bubbled below the solvent surface using a balloon. After one h, reaction mixture was filtered. Final product was isolated after addition of 2 drops of concentrated HCl in 92% yield. HRMS calcd for C₂₃H₂₁N₄O₃ (M)+, 401.1608; found, 401.1608. ¹H NMR (400 MHz, DMSO-d₆) δ ppm 1.31 (t, J=7.15 Hz, 3H) 4.33 (q, J=6.97 Hz, 2H) 6.61 (br. s., 1H) 7.12 (d, J=7.70 Hz, 2H) 7.19 (t, J=7.30 Hz, 1H) 7.43 (t, J=7.70 Hz, 2H) 7.64 (br. s., 1H) 7.79 (ddd, J=8.40, 4.00 Hz, 1H) 7.87-8.13 (m, 3H) 8.75 (d, J=8.43 Hz, 1H) 10.46 (br. s., 1H).

4-((3-Amino-5-phenoxyphenyl)amino)quinazoline-2-carboxylic acid (6a). Compound 5a (30 mg, 0.08 mmol) was stirred at 50° C. ethanol in presence of 0.5 mL of 4N NaOH. Ester cleavage was monitored by LCMS. Compound 6a was isolated as the HCl salt after completion of the reaction by addition of 2 drops of concentrated HCl in 90% yield. HRMS calcd for C₂₁H₁₇N₄O₃ (M)+, 373.1295; found, 373.1300. ¹H NMR (400 MHz, DMSO-d₆) δ ppm 6.22 (br. s., 1H) 7.13 (t, J=8.80 Hz, 1H) 6.95-7.19 (m, 3H) 7.19-7.31 (m, 1H) 7.39 (t, J=7.88 Hz, 2H) 7.74 (m, J=8.10, 5.90 Hz, 1H) 7.89-8.04 (m, 2H) 8.64 (d, J=8.07 Hz, 1H) 10.21 (br. s., 1H).

4-((3-Nitro-5-phenoxyphenyl)amino)quinazoline-2-carboxylic acid (7a). Compound 1a (34 mg, 0.08 mmol) was stirred at 50° C. ethanol in presence of 0.5 mL of 4N NaOH. Ester cleavage was monitored by LCMS. 7a was isolated as the HCl salt after completion of the reaction by addition of 2 drops of concentrated HCl in 94% yield. HRMS calcd for C₂₁H₁₄N₄O₅Na (M+Na)+, 425.0856; found, 425.0859. ¹H NMR (400 MHz, DMSO-d₆) δ ppm 7.14-7.36 (m, 3H) 7.48 (t, J=6.80 Hz, 2H) 7.56-7.68 (m, 2H) 7.69-7.80 (m, 1H) 7.81-7.95 (m, 1H) 8.48-8.72 (m, 2H) 8.81 (s, 1H) 10.17 (s, 1H).

4-((3-(2-Bromophenoxy)-5-nitrophenyl)amino)quinazoline-2-carboxylic acid (7g). Compound 1g (50 mg, 0.1 mmol) was stirred at 50° C. ethanol in presence of 0.5 mL of 4N NaOH. Ester cleavage was monitored by LCMS. 7g was isolated as the HCl salt after completion of the reaction by addition of 2 drops of concentrated HCl in 92% yield. HRMS calcd for C₂₁H₁₄N₄O₅ (M+Na)+, 402.096421; found, 455.1511. ¹H NMR (400 MHz, DMSO-d₆) δ ppm 7.19 (s, 1H) 7.27 (t, J=8.30 Hz, 1H) 7.38 (d, J=7.31Hz, 1H) 7.51 (t, J=7.30 Hz, 1H) 7.56-7.69 (m, 1H) 7.82 (d, J=7.31Hz, 2H) 7.98-8.16 (m, 1H) 8.43-8.61 (m, 2H) 8.90 (br. s., 1H) 10.13 (br. s., 1H).

N-Ethyl-4-((3-nitro-5-phenoxyphenyl)amino)quinazoline-2-carboxamide (8a). To a solution of compound 7a (20 mg, 0.05 mmol), triethylamine (14 μL, 0.1 mmol) and HATU (21 mg, 0.055 mmol) in 1 mL of DMF was added 2M Ethylamine in THF (49 μL, 0.1 mmol) and stirred at rt and monitored by LC/MS. Upon completion, the reaction mixture was concentrated in vacuo and purified on silica with a gradient system of 5:95 methanol:DCM to 10:90 methanol:DCM to give 19.1 mg in 89% yield. ¹H NMR (400 MHz, DMSO-d₆) δ ppm 1.16 (t, J=7.00 Hz, 3H) 3.03-3.22 (m, 2H) 7.23 (d, J=8.07 Hz, 2H) 7.28 (t, J=7.52Hz, 1H) 7.43 (t, J=2.02Hz, 1H) 7.50 (t, J=7.70 Hz, 2H) 7.78 (ddd, J=8.34, 5.96, 2.20 Hz, 1H) 7.93-8.02 (m, 2H) 8.34 (t, J=1.83 Hz, 1H) 8.63 (d, J=7.70 Hz, 2H) 8.92 (t, J=1.80 Hz, 1H) 10.32 (s, 1H).

4-((3-nitro-5-phenoxyphenyl)amino)quinazoline-2-carboxamide (9a). Compound 1a (20 mg, 0.05 mmol) was stirred in 7N ammonia solution in methanol in a pressure vessel at 90° C. overnight. 9a was purified by column chromatography (40 to 90% ethyl acetate in hexanes gradient). HRMS calcd for C₂₁H₁₅N₅O₄Na (M+Na)+, 424.1016; found, 424.1017. ¹H NMR (300 MHz, DMSO-d₆) δ ppm 7.24 (d, J=8.25 Hz, 2H) 7.28 (t, J=7.20 Hz, 1H) 7.44 (s, 1H) 7.51 (t, J=7.70 Hz, 2H) 7.68-7.84 (m, 1H) 7.87-8.06 (m, 2H) 8.33 (s, 1H) 8.63 (d, J=7.98 Hz, 1H) 8.85 (s, 1H) 10.31 (br. s., 1H).

Methyl 4-((3-nitro-5-phenoxyphenyl)amino)quinazoline-2-carboxylate (10a). Compound 7a (20 mg, 0.05 mmol) was converted to the acid chloride in neat thionyl chloride and 3 drops of DMF at 65° C. Reaction was monitored by TLC and the mixture was taken to dryness after completion Anhydrous methanol was added immediately and stirred at 30° C. for 10 minutes. 10a was purified by normal phase column chromatography (40 to 90% ethyl acetate in hexanes gradient) in 38% isolated yield. HRMS calcd for C₂₂H₁₇N₄O₅ (M)+, 417.1193; found, 417.1195. ¹H NMR (300 MHz, DMSO-d₆) δ ppm 3.88 (s, 3H) 7.26 (t, J=7.98 Hz, 1H) 7.21 (d, J=7.98 Hz, 2H) 7.36-7.56 (m, 3H) 7.77-7.86 (m, 1H) 7.99 (m, J=3.90 Hz, 2H) 8.30 (br. s., 1H) 8.64 (d, J=8.80 Hz, 1H) 9.07 (s, 1H) 10.36 (s, 1H).

Isopropyl 4-((3-nitro-5-phenoxyphenyl)amino)quinazoline-2-carboxylate (11a). Compound 7a (20 mg, 0.05 mmol) was converted to the acid chloride in neat thionyl chloride with 3 drops of DMF at 65° C. Reaction was monitored by TLC and the mixture was taken to dryness after completion Anhydrous isopropyl alcohol was added immediately and stirred at 30° C. for 10 minutes. 11a was purified by normal phase column chromatography (40 to 90% ethyl acetate in hexanes gradient) in 32% isolated yield. HRMS calcd for C₂₄H₂₁N₄O₅ (M)+, 445.1506; found, 445.1507. ¹H NMR (300 MHz, DMSO-d₆) δ ppm 1.35 (d, J=6.33 Hz, 6H) 5.04-5.36 (m, 1H) 7.28 (t, J=7.20 Hz, 1H) 7.21 (d, J=8.53 Hz, 2H) 7.44 (s, 2H) 7.50 (t, J=7.70 Hz, 1H) 7.83 (m, J=8.10, 3.40 Hz, 1H) 7.97-8.07 (m, 2H) 8.46 (s, 1H) 8.79 (d, J=8.25 Hz, 1H) 9.09 (s, 1H) 10.56 (s, 1H).

Isopentyl 4-((3-nitro-5-phenoxyphenyl)amino)quinazoline-2-carboxylate (12a). Compound 7a (20 mg, 0.05 mmol) was converted to acid chloride in neat thionyl chloride and 3 drops of DMF at 65° C. Reaction was monitored by TLC and the mixture was taken to dryness after completion Anhydrous isopentyl alcohol was added immediately and stirred at 30° C. for 10 minutes. 12a was purified by normal phase column chromatography (40 to 90% ethyl acetate in hexanes gradient) in 30% isolated yield with purity >90%. HRMS calcd for C₂₆H₂₅N₄O₅ (M)+, 473.1819; found, 473.1820. ¹H NMR (500 MHz, DMSO-d₆) δ ppm 0.89 (d, J=6.60 Hz, 6H) 1.68-1.77 (m, 1H) 4.36 (t, J=6.85 Hz, 2H) 7.22 (d, J=7.34 Hz, 2H) 7.28 (t, J=7.60 Hz, 1H) 7.41 (s, 1H) 7.50 (t, J=7.60 Hz, 2H) 7.83 (m, J=4.60 Hz, 1H) 8.01 (d, J=3.67 Hz, 2H) 8.45 (s, 1H) 8.66 (d, J=8.31Hz, 1H) 8.95 (s, 1H) 10.37 (s, 1H).

Isopropyl 4-((3-(2-bromophenoxy)-5-nitrophenyl)amino)quinazoline-2-carboxylate (13g). Compound 7g (20 mg, 0.05 mmol) was converted to acid chloride in neat thionyl chloride and 3 drops of DMF at 65° C. Reaction was monitored by TLC and the mixture was taken to dryness after completion Anhydrous isopropyl alcohol was added immediately and stirred at 30° C. for 10 minutes. 13g was purified by normal phase column chromatography (40 to 90% ethyl acetate in hexanes gradient) in 42% isolated yield. HRMS calcd for C₂₄H₁₉BrN₄O₅ (M+Na)+, 545.0430; found, 545.0431. ¹H NMR (500 MHz, DMSO-d₆) δ ppm 1.36 (d, J=6.11 Hz, 6H) 5.15-5.26 (m, 1H) 7.29 (t, J=7.70 Hz, 1H) 7.33-7.40 (m, 2H) 7.52 (t, J=7.80 Hz, 1H) 7.69 (d, J=10.27 Hz, 1H) 7.76-7.91 (m, 1H) 7.94-8.11 (m, 2H) 8.36 (s, 1H) 8.66 (d, J=8.31Hz, 1H) 9.08 (s, 1H) 10.37 (s, 1H).

### REFERENCES

(Experimental Section). Chan, M.; Hayashi, T.; Mathewson, R. D.; Nour, A.; Hayashi, Y.; Yao, S.; Tawatao, R. I.; Crain, B.; Tsigelny, I. F.; Kouznetsova, V. L.; Messer, K.; Pu, M.; Con, M.; Carson, D. A.; Cottam, H. B. J. Med. Chem. 2013, 56, 4206; Pu, M.; Hayashi, T.; Cottam, H.; Mulvaney, J.; Arkin, M.; Con, M.; Carson, D.; Messer, K. Stat. Med. 2012, 31, 4175; Hajjar, A. M.; Ernst, R. K.; Fortuno, E. S., 3rd; Brasfield, A. S.; Yam, C. S.; Newlon, L. A.; Kollmann, T. R.; Miller, S. I.; Wilson, C. B. PLoSPathog. 2012, 8, e1002963; Wu, C. C.; Hayashi, T.; Takabayashi, K.; Sabet, M.; Smee, D. F.; Guiney, D. D.; Cottam, H. B.; Carson, D. A. Proc. Natl. Acad. Sci. U.S.A. 2007, 104, 3990; Chan, M.; Hayashi, T.; Kuy, C. S.; Gray, C. S.; Wu, C. C.; Con, M.; Wrasidlo, W.; Cottam, H. B.; Carson, D. A. Bioconjug. Chem. 2009, 20, 1194; Hayashi, T.; Rao, S. P.; Takabayashi, K.; Van Uden, J. H.; Kornbluth, R. S.; Baird, S. M.; Taylor, M. W.; Carson, D. A.; Catanzaro, A.; Raz, E. Infect. Immun. 2001, 69, 6156; Crozat, K.; Georgel, P.; Rutschmann, S.; Mann, N.; Du, X.; Hoebe, K.; Beutler, B. Mamm. Gen. 2006, 17, 398.

What is claimed is:

1. A compound having the structure of Formula (I): 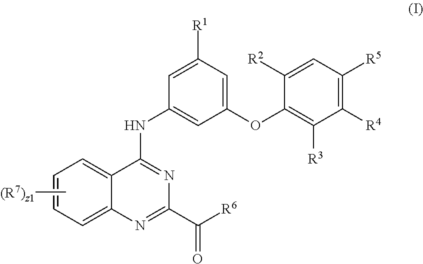
or a pharmaceutically acceptable salt thereof,
wherein
R¹ and R² are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³, R⁴ and R⁵ are independently hydrogen, halogen, —CN, —SH, —OH, —COOH, —NH₂, —CONH₂, —NO₂, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or wherein R³ and R⁴ are optionally joined together to form a substituted or unsubstituted cycloalkyl, substituted or unsubstituted heterocycloalkyl, substituted or unsubstituted aryl or substituted or unsubstituted heteroaryl, or wherein R⁴ and R⁵ are optionally joined together to form a substituted or unsubstituted cycloalkyl, substituted or unsubstituted heterocycloalkyl, substituted or unsubstituted aryl or substituted or unsubstituted heteroaryl;
R⁶ is —OR⁹, —N(R⁸)(R⁹), substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is independently halogen, —CN, —SH, —OH, —COOH, —NH₂, —CONH₂, —NO₂, —CF₃, —CCl₃, -L¹-R¹³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently hydrogen or substituted or unsubstituted alkyl;
R⁹ is independently hydrogen, substituted or unsubstituted alkyl, or a hydrophilic polysaccharide;
L¹ is substituted or unsubstituted alkylene or substituted or unsubstituted heteroalkylene;
R¹³ is substituted or unsubstituted cycloalkyl, substituted or unsubstituted heterocycloalkyl, substituted or unsubstituted aryl, or substituted or unsubstituted heteroaryl; and
z1 is an integer from 0 to 4.

2. The compound of claim 1, wherein R¹ is —NO₂.

3. The compound of claim 2, wherein R², R³, R⁴, and R⁵ are independently hydrogen, halogen, —CF₃, or substituted or unsubstituted alkyl.

4. The compound of claim 2, wherein
R³ is hydrogen, halogen, —CF₃, or unsubstituted C₁-C₅ alkyl; and
R², R⁴, and R⁵ are independently hydrogen or unsubstituted C₁-C₅ alkyl.

5. The compound of claim 2, wherein R⁶ is —OR⁹, —N(R⁸)(R⁹), substituted or unsubstituted alkyl, substituted or unsubstituted heteroalkyl.

6. The compound of claim 5, wherein R⁸ is hydrogen and R⁹ is independently hydrogen or substituted or unsubstituted C₁-C₅ alkyl.

7. The compound of claim 1 having the structure of Formula (III): 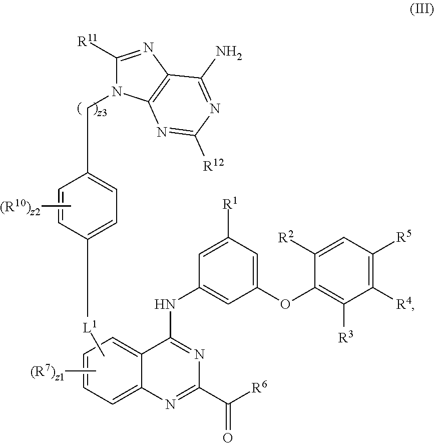
or a pharmaceutically acceptable salt thereof,
wherein
R¹⁰ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ is —SR^{11A} or —OR^{11A};
R^{11A} is hydrogen, substituted or unsubstituted alkyl, substituted or unsubstituted heteroalkyl, substituted or unsubstituted heterocycloalkyl, substituted or unsubstituted cycloalkyl, substituted or unsubstituted aryl, or substituted or unsubstituted heteroaryl;
R¹² is hydrogen, halogen, —NO₂, —OH, —SH, —CN, —COOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
z2 is an integer from 0 to 4; and
z3 is an integer from 1 to 10.

8. A method of modulating a Toll-like receptor protein, said method comprising contacting the Toll-like receptor protein with a compound of claim 1.

9. The method of claim 8, wherein said Toll-like receptor protein is TLR4.

10. A method of treating or preventing a disease in a subject in need thereof, said method comprising administering to said subject a therapeutically effective amount of a compound of claim 1 to modulate an immune response in said subject.

11. The method of claim 10, wherein said compound is administered to said subject as a pharmaceutical composition or a vaccine composition.

12. The method of claim 10, wherein said disease is cancer.

13. The method of claim 12, wherein said modulating of said immune response in said subject having cancer comprises increasing an immune response to a cancer cell relative to the absence of said compound.

14. The method of claim 10, wherein said disease is an infectious disease.

15. The method of claim 14, wherein said modulating of said immune response in said subject having an infectious disease comprises increasing an immune response to a pathogen relative to the absence of said compound.

16. The method of claim 10, wherein said disease is an autoimmune disease.

17. The method of claim 16, wherein said modulating of said immune response in said subject having an autoimmune disease comprises decreasing an immune response to an endogenous antigen relative to the absence of said compound.

18. The method of claim 17, wherein said endogenous antigen is associated with said autoimmune disease.

19. The method of claim 10, wherein said disease is an inflammatory disease.

20. The method of claim 19, wherein said modulating of said immune response in said subject having an inflammatory disease comprises decreasing an immune response to said inflammation in said subject.


SEQUENCE LISTING (11 sequences)

SEQ ID NO: 1 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 2 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 3 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 4 - protein, 799 aa, Homo sapiens
MELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 5 - protein, 639 aa, Homo sapiens
MPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 6 - protein, 858 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEILDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELYSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSFSGVSLFSLSTEGCDEEEVLKSLKFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDLQDVGWFLHKLSQQILKKEKEKKKDNNIPLQTVATIS

SEQ ID NO: 7 - protein, 1049 aa, Homo sapiens
MVFPMWTLKRLILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 8 - protein, 1059 aa, Homo sapiens
MKESSLQNSSCSLGKETKKENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 9 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 10 - protein, 605 aa, Homo sapiens
EPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNK

SEQ ID NO: 11 - protein, 142 aa, Homo sapiens
QKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN